(19) United States
(12) Patent Application Publication
(10) Pub. No.: US 2008/0233220 A1
(43) Pub. Date: Sep. 25, 2008

(54) Further Medical Use Of A Botanical Drug Or Dietary Supplement

(76) Inventors: Shouming Zhong, Oxford (GB); Hongwen Yu, Oxforshire (GB)

Correspondence Address: DICKINSON WRIGHT PLLC, 38525 WOODWARD AVENUE, SUITE 2000, BLOOMFIELD HILLS, MI, 48304-2970, US

(21) Appl. No.: 12/063,610

(22) PCT Filed: Aug. 8, 2006
(86) PCT No.: PCT/GB06/02948

§ 371 Date: Feb. 12, 2008

(30) Foreign Application Priority Data
Aug. 12, 2005 (GB) 0516601.2

(51) Int. Cl. A61K 36/537; A61K 31/357; A61K 31/704; A61K 31/343; A61K 31/09; A61P 1/16

(52) U.S. Cl. 424/746; 514/452; 514/26; 514/468; 514/721

(57) ABSTRACT

The present invention relates to further medical uses for a botanical drug or dietary supplement consisting essentially of four botanical drug substances, optionally formulated with excipients. The botanical raw materials, botanical drug substances or botanical ingredients used are from a species of each of the genera (a) Silybum; (b) Astragalus or Hedysarum; (c) Salvia; and (d) Schisandra.

### TECHNICAL FIELD OF THE INVENTION

The present invention relates to a botanical drug or dietary supplement for use in the treatment of patients suffering from liver disease, be it HCV-associated liver disease, HBV-associated liver disease or liver disease caused by drug abuse, alcohol abuse or as a result of diabetes. More particularly, it relates to the use of a botanical drug consisting essentially of four botanical drug substances, optionally formulated with excipients.

Specifically, the invention relates to the finding that the claimed composition exhibited activity in clinical trials which suggested that in addition to, or as an alternative to, the anti-viral activity disclosed in applicants' earlier application PCT/GB2005/000559 (unpublished at the time of filing) the composition additionally shows promise as:
1. a candidate for use as an adjunct therapy with interferon and ribivarin (or other immuno-modulator/antiviral drug combinations);
2. a candidate for use in the treatment of patients who do not respond to interferon and ribivarin (or other immuno-modulator/antiviral drug combinations) treatment;
3. a candidate for stand alone treatment for HCV or HBV-associated liver disease;
4. a candidate for treating alcoholic livers; and
5. a candidate for treating fatty livers.

### BACKGROUND OF THE INVENTION

The applicant has developed a botanical drug consisting essentially four botanical drug substances. Unusually, they comprise an extract of silybum (a Western herb) and extracts of only three Chinese herbs. The applicant believes such a combination of non indigenous herbs is particularly unusual.

In their earlier application PCT/GB2005/000559, which document is incorporated by reference, they proposed that the botanical drug would be beneficial in alleviating the symptoms of Hepatitis C, and for inhibiting the activity of the causative Hepatitis C virus based upon its activity in a Replicon assay (Example 3).

### DEFINITIONS

In the specification the following definitions, taken from the U.S. Department of Health and Human Services, Food and Drug Administration, Center for Drug Evaluation and Research (CDER), August 2000 Guidance for Industry, Botanical Drug Products, are intended:

Active Constituent The chemical constituent in a botanical raw material, drug substance, or drug product that is responsible for the intended pharmacological activity or therapeutic effect.

Botanical Product; Botanical: A finished, labelled product that contains vegetable matter, which may include plant materials (see below), algae, macroscopic fungi, or combinations of these. Depending in part on its intended use, a botanical product may be a food, drug, medical device, or cosmetic.

Botanical Drug Product; Botanical Drug: A botanical product that is intended for use as a drug; a drug product that is prepared from a botanical drug substance. Botanical drug products are available in a variety of dosage forms, such as solutions (e.g., teas), powders, tablets, capsules, elixirs, and topicals.

Botanical Drug Substance: A drug substance derived from one or more plants, algae, or macroscopic fungi. It is prepared from botanical raw materials by one or more of the following processes: pulverization, decoction, expression, aqueous extraction, ethanolic extraction, or other similar process. It may be available in a variety of physical forms, such as powder, paste, concentrated liquid, juice, gum, syrup, or oil. A botanical drug substance can be made from one or more botanical raw materials (see Single-Herb and Multi-Herb botanical drug substance or product). A botanical drug substance does not include a highly purified or chemically modified substance derived from natural sources.

Botanical Ingredient: A component of a botanical drug substance or product that originates from a botanical raw material.

Botanical Raw Material: Fresh or processed (e.g., cleaned, frozen, dried, or sliced) part of a single species of plant or a fresh or processed alga or macroscopic fungus.

Chromatographic Fingerprint: A chromatographic profile of a botanical raw material or drug substance that is matched qualitatively and quantitatively against that of a reference sample or standard to ensure the identity and quality of a batch and consistency from batch to batch.

Dietary Supplement: [A] product (other than tobacco) intended to supplement the diet that bears or contains one or more of the following dietary ingredients: (A) a vitamin; (B) a mineral; (C) an herb or other botanical; (D) an amino acid; (E) a dietary substance for use by man to supplement the diet by increasing the total dietary intake; or (F) a concentrate, metabolite, constituent, extract, or combination of any ingredient described in clause (A), (B), (C), (D), or (E); (2) means a product that (A) is intended for ingestion in a form described in section 411(c)(1)(B)(i) [of the FD&C Act]; or complies with section 411(c)(1)(B)(ii); is not represented for use as a conventional food or as a sole item of a meal or the diet; and is labelled as a dietary supplement; and (3) does (A) include an article that is approved as a new drug under section 505 or licensed as a biologic under section 351 of the Public Health Service Act (42 U.S.C. 262) and was, prior to such approval, certification, or license, marketed as a dietary supplement or as a food unless [FDA] has issued a regulation, after notice and comment, finding that the article, when used as or in a dietary supplement under the conditions of use and dosages set forth in the labelling for such dietary supplement, is unlawful under section 402(f); and (1) not include (i) an article that is approved as a new drug under section 505, certified as an antibiotic under section 507, or licensed as a biologic under section 351 of the Public Health Service Act (42 U.S.C. 262), or (ii) an article authorized for investigation as a new drug, antibiotic, or biological for which substantial clinical investigations have been instituted and for which the existence of such investigations has been made public, which was not before such approval, certification, licensing, or authorization marketed as a dietary supplement or as a food unless [FDA], in [its] discretion, has issued a regulation, after notice and comment, finding that the article would be lawful under this Act_(21 U.S.C. 321(ff)).

Dosage Form: A pharmaceutical product type, for example, tablet, capsule, solution, or contains a drug ingredient (substance) generally, but not necessarily, in association with excipients.

Drug: Means (A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals; and (D) articles intended for use as a component of any articles specified in clause (A), (B), or (C). A food or dietary supplement for which a claim, subject to sections 403(r)(1)(B) and 403(r)(3) [of the FD&C Act] or sections 403(r)(1)(B) and (r)(5)(D), is made in accordance with the requirements of section 403(r) is not a drug solely because the label or the labelling contains such a claim. A food, dietary ingredient, or dietary supplement for which a truthful and not misleading statement is made in accordance with section 403(r)(6) is not a drug under clause (C) solely because the label or the labelling contains such a statement_(21 U.S.C. 321(g)(1)).

Drug Substance: An active ingredient that is intended to furnish pharmacological activity or other direct effect in the diagnosis, cure, mitigation, treatment, or prevention of disease or to affect the structure or any function of the human body (21 CFR 314.3(b)).

Drug Product The dosage form in the final immediate packaging intended for marketing.

Food: The term food means (1) articles used for food or drink, (2) chewing gum, and (3) articles used for components of such articles (21 U.S.C. 321 (f)).

Formulation: A formula that lists the components (or ingredients) and composition of the dosage form. The components and composition of a multi-herb botanical drug substance should be part of the total formulation.

Marker: A chemical constituent of a botanical raw material, drug substance, or drug product that is used for identification and/or quality control purposes, especially when the active constituents are not known or identified.

Multi-Herb (Botanical Drug) Substance or Product: A botanical drug substance or drug product that is derived from more than one botanical raw material, each of which is considered a botanical ingredient. A multi-herb botanical drug substance may be prepared by processing together two or more botanical raw materials, or by combining two or more single-herb botanical drug substances that have been individually processed from their corresponding raw materials. In the latter case, the individual single-herb botanical drug substances may be introduced simultaneously or at different stages during the manufacturing process of the dosage form.

Plant Material: A plant or plant part (e.g., bark, wood, leaves, stems, roots, flowers, fruits, seeds, berries, or parts thereof) as well as exudates.

Single-Herb (Botanical Drug) Substance or Product: A botanical drug substance or drug product that is derived from one botanical raw material. Therefore, a single-herb substance or product generally contains only one botanical ingredient.

In addition the terms:

Consisting essentially is intended to refer back only to the presence of the botanical raw materials and their derivatives and excludes the presence of e.g. excipients used in the formulation;

Treatment is intended to refer to both symptomatic relief and/or activity against the causative factor.

The composition of the present invention is unusual in that it comprises a combination of a Western herb and a small number (only three) Chinese herbs.

In Traditional Chinese Medicine (TCM), HCV infection is regarded as causing the following pathological changes in the body:
accumulation of toxin and heat in the blood;
consumption of vital energy and body fluid;
stagnation of blood; and
injury of liver and spleen function.

In order to address these different aspects existing TCM plant based formulations for e.g. HCV treatment usually contain many ingredients, typically ten or more. For practical purposes it would clearly be desirable and advantageous to minimise the number of botanical ingredients or botanical drug substances without in any way compromising therapeutic efficacy.

This unique combination of a Western Herb and Chinese herbs brings with it hither to unknown issues of safety as well as efficacy.

The prior art, of course, makes reference to the use of the Western herb (Silybum) alone as taught by, for example, the following:

Rodriguez-Perez. et al: “The effect of Silybum marianum on the viral load of Hispanic patients with chronic hepatitis C” American Journal of Gastroenterology, Vol 97, No. 9, which teaches that Silybum marianum may have a protective effect in the inflammatory response to hepatitis C virus. This supposition was based on the observation of a lack of increase in the liver enzymes in subjects taking this herb. The enzymes measured were AST and ASL.

Chavez, Mary “Treatment of hepatitis C with milk thistle?” Journal of Herbal Pharmacotherapy, Vol 1, No 3, P. 79-90. This document discloses a number of studies of patients with various chronic diseases. It notes that in one study of patients with mild alcoholic liver disease (evidenced by elevated AST and ALT levels) treatment with silymarin resulted in a significant change in these enzyme levels. In another study of patients with biopsy confirmed cirrhosis, patients treated with silymarin lived longer.

Significantly the paper concludes by commenting that “The current scientific evidence supporting the use of silymarin for treatment of chronic hepatitis C is equivocal . . . . Milk thistle extract does not decrease viral load.”

Fogden et al “Alternative medicines and the liver” Liver International 1 Aug. 2003, Vol 23, no 4 teaches the use of silymarin in the treatment of hepatobiliary disease due to its anti inflammatory activity. Its effect on alcohol related liver disease and cirrhosis as also noted although it goes on to note that evidence e.g. effects on survival or biochemical variables is mixed.

Thus, whilst Silymarium has been used alone with mixed results there is a clear need for a product which has a capability to provide multiple benefits.

Fogden et al also discloses the use of herbal mixtures most of which are particularly complex comprising a large number of herbs.

For the development of Western medicines this raises issues of:
Quality control/standardisation, and
Drug interaction (and safety)

The present invention is unusual in that it combines a Western herb with a limited number of Chinese herbs. Such a combination would not be obvious to the average person skilled in the art.

In Chinese medicine it is usual to use a relatively large number of herbs. Thus typical of the art are:

CN 1,071,581A, which describes an anti hepatic including seven herbs including salvia miltiorrhiza, astragalus menbranaceus and magnolia vine.

CN 1,371,713A, which discloses a twenty herb combination including salvia root, astragalus root and schisandra berry.

CN 1,393,255A, (abstract) which combines nineteen Chinese medicinal materials including astragalus root and red sage root.

CN 1,166,342A, (which discloses five named herbs including red sage and a number of “other” unnamed herbs

WO 02/32444A discloses a fifteen ingredient product of which the four core ingredients include schisandra. This document, in discussing the related art, makes reference to other Chinese herbal compositions including Gandezhi a capsule containing scutellaria and salvia root for lowering transaminase levels and Wurzi a fructus schisandra extract for lowering GTP levels.

Other prior art includes:

CN 1053225 which discloses a twelve herb medicine including schisandra fruit, and

CN 1151312 which discloses a ten herb medicine including schisandra fruit.

Thus, the prior art generally comprises complex Chinese herbal mixtures. There is no suggestion in the art to combine a Western herb with a limited number of Chinese herbs and any real indication that such a combination would be safe and efficacious. Let alone one which could be formulated to produce a product which can be formulated to form a suspension in a small volume of liquid as is disclosed.

Surprisingly the applicant has found that a combination of only four plant species demonstrates activity against Hepatitis C virus and additionally in a clinical setting shows activity in terms of primary outcomes, secondary measures as well as showing a good safety profile. It is these clinical findings which form the basis of the broader medical applications for this unique combination.

### SUMMARY OF THE INVENTION

According to a first aspect of the present invention there is provided the use of a botanical drug or dietary supplement consisting essentially of botanical raw materials, botanical drug substances or botanical ingredients from each of:
   (a) The fruit of Silybum marianum;
   (b) The root of Astragalus membranaceus var mongholicus or Hedysarum polybotrys;
   (c) The root of Salvia miltiorrhiza, Salvia bowleyana or Salvia przewalskii; and
   (d) The fruit of Schisandra chinensis or Schisandra sphenanthera
      in the manufacture of a medicament for use in the treatment or prevention of one or more of the following:
   i) Liver inflammation associated with hepatitis B virus;
   ii) Liver inflammation associated with alcohol abuse;
   iii) Metabolic disorders associated with the liver, including for example, diabetes and metabolic syndrome X;
   iv) Fatty liver;
   v) Treating patients who are non responsive to immuno-modulatory/antiviral combination therapies such as, interferon/ribovarin;
   vi) As an adjunct therapy to combination therapies such as, interferon/ribovarin
   vii) HCV associated liver disease;
   viii) Hepatitis; fibrosis, cirrhosis or hepatocellular carcinoma
   ix) Treatment to reduce raised liver enzyme levels associated with chemotherapy.

The fruit of Silybum marianum is known in TCM as Sui Fei Ji and in Western Europe as milk thistle fruit.

The root of Astragalus membranaceus var mongholicus is known in TCM as Huang Qi and in Western Europe as Astragalus root. The root of Hedysarum polybotyrs is known in TCM as Hong Qi The Astragalus species and Hedysarum species disclosed in this application may be used interchangeably in TCM.

The root of Salvia miltiorrhiza is known in TCM as Dan Shen and in Western Europe as Chinese sage root. Alternatively Salvia bowleyana or Salvia przewalskii may be used. The Salvia species disclosed in this application may be used interchangeably in TCM

The fruit of Schisandra chinensis is known in TCM as Wu Wei Zi, and in Western Europe as Schisandra fruit. Alternatively Schisandra sphenanthera may be used. The Schisandra species disclosed in this application may be used interchangeably in TCM

In a preferred embodiment the plant species are:
a) Silybum marianum;
b) Astragalus membranaceus var mongholicus;
c) Salvia miltiorrhiza; and
d) Schisandra chinensis.

In alternative embodiments the Astragalus membranaceus var mongholicus may be substituted with Hedysarum polybotyrs.

A particularly preferred composition of the invention comprises: Sui Fei Ji; Dan Shen; Wu Wei Zi; and Huang Qi.

Whilst in a favoured embodiment the invention takes the form of a botanical drug, consisting essentially of botanical drug substances of each of the four plant species in further embodiments the botanical drug may consist essentially of botanical ingredients of each of the species. Where the product is a dietary supplement the four plant species may additionally be in the form of botanical raw materials.

In the case of a botanical drug there may be present, in addition to the botanical drug substances, pharmaceutically acceptable excipients.

In the case of a dietary supplement there may be present in addition to the botanical raw materials, botanical drug substances or botanical ingredients one or more dietetically acceptable excipients.

The present invention also provides a method of treatment or dietary supplementation which comprises administering to a human a composition of the invention in an amount sufficient to treat or prevent inflammatory liver disease, hepatitis, fibrosis, cirrhosis and hepatocellular carcinoma.

In particular administration of the composition has been demonstrated to improve primary outcome in patients by way of quality of life scores (SF36 and FFS); and by way of secondary measures reduce liver inflammation. These secondary measures include:
Lowered GGT (gamma glutamyl amino transferase),
Lowered ALT, (alanine amino transferase) and
Lowered AST levels (aspartyl amino transferase) as well as
Maintained total bilirubin levels.

Additionally, the safety profile (effect on haemoglobin, white blood cells, blood platelets, creatine and blood glucose) indicate it could be used in combination therapies with for example interferon and ribivarin.

The plant materials may be employed in the composition of the invention in any suitable form. This may for instance be as crude plant material, which is either fresh or dried, or as an extract of fresh or dried plant material, i.e. a botanical drug substance. The extract is preferably a total plant extract defined with reference to one or more chemical markers although defined fractions and botanical ingredients may also be used. The extract, most usually a botanical drug substance, is typically dried and used in powder form, most preferably as a lyophilised extract.

When botanical drug substance is used it is preferably pulverized. In this embodiment the botanical drug substance is dried and ground to a powder. The resulting powder of the or each botanical drug substance is then conveniently mixed together to form a plant based composition of the invention in powder form. This powder can be administered directly, for instance by being dispersed in a liquid for human subjects to drink. Alternatively the powder can be processed into any other conventional dosage form such as capsules, tablets or granules. In a preferred embodiment the applicant has developed a suspension formulation which is suspendable in a relatively small volume of a cold liquid, such as water. Typically the suspension formulation can be suspended in less than 50 ml, more typically less than 25 ml of water. Preferably the packaged medicament is supplied with a dispensing container.

A botanical drug substance, for instance a total extract, may be prepared by any conventional technique known for the extraction of ingredients from botanical materials. These include solvent extraction including supercritical fluid extraction using a liquefied gas such as carbon dioxide. In one embodiment the extracts are ethanolic extracts, such as those obtained using 70% ethanol. The extracts are most preferably standardised extract, for instance a standardised total extract. The preferred standardised total extracts are pharmaceutical grade extracts.

An extract is typically prepared by immersing or macerating or refluxing fresh or dry plant material, for instance powdered dry plant material, in a suitable solvent; separating solid residue from the solution, removing the solvent from the solution; and recovering the resulting concentrates.

If desired a liquid extract may be dried before being formulated into a botanical drug or dietary supplement of the invention, for instance by spray drying or by freeze drying (lyophilisation). In that case the dried extract of one or more of the constituent plant species of the composition of the invention may be mixed with pulverized dried plant material of one or more of the other constituent plant species, to form a powder for direct administration to human subjects or for encapsulation or tabletting into unit dosage forms. Alternatively the extract may be used directly without prior drying.

The botanical raw materials or botanical drug substances or botanical ingredients may be combined together using any conventional technique that is suitable for ingredients of this type. When the botanical raw materials, drug substances or botanical ingredients are all in dry form they are conveniently mixed together, for instance by hand or by means of a mechanical mixer. A mixing procedure of this type may also be suitable if some, but not all, of the components of the plant based composition are in dry form.

The Silybum marianum is preferably employed in the form of a pharmaceutical grade extract that can be obtained commercially from, for example, an Italian manufacturer, Indena. The pharmaceutical grade Silybum marianum extract manufactured by Indena is standardized for silymarin content of no less than 30% weight percent by HPLC. The pharmaceutical grade extract must pass extensive safety and efficacy procedures. Preferably, when employed in the practice of the present invention the Silybum marianum extract has a minimum silymarin content of at least 30% by HPLC analysis.

The Astragalus membranaceus var mongholicus is preferably employed in the form of a pharmaceutical grade extract that can be obtained commercially from, for example, a Chinese manufacturer, the Institute of Medicinal Plant Development, Haiding District, Xibeiwang, Beijing 100094, China. Pharmaceutical grade Astragalus membranaceus var mongholicus extract manufactured in China is standardized for an Astragaloside IV content of about 0.4 weight percent. The pharmaceutical grade extract must pass extensive safety and efficacy procedures. Preferably, when employed in the practice of the present invention the Astragalus membranaceus var mongholicus extract has an Astragaloside IV content of from 0.1 to about 10 weight percentage. Preferably, the Astragalus membranaceus var mongholicus extract used in the present invention has a minimum Astragaloside IV content of at least 0.4 percent.

The Salvia miltiorrhiza is preferably employed in the form of a pharmaceutical grade extract that can be obtained commercially from, for example, a Chinese manufacturer, the Institute of Medicinal Plant Development, Haiding District, Xibeiwang, Beijing 100094, China Pharmaceutical grade Salvia miltiorrhiza extract manufactured in China is standardized for a Tanshinone IIa content of about 1.5 weight percent. The pharmaceutical grade extract must pass extensive safety and efficacy procedures. Preferably, when employed in the practice of the present invention the Salvia miltiorrhiza extract has a Tanshinone IIa content of from 1.5 to about 50% weight percentage. Preferably, the Salvia miltiorrhiza extract used in the present invention has a minimum Tanshinone IIa content of at least 2.0 percent.

The Schisandra chinensis is preferably employed in the form of a pharmaceutical grade extract that can be obtained commercially from, for example, a Chinese manufacturer, the Institute of Medicinal Plant Development, Haiding District, Xibeiwang, Beijing 100094, China. Pharmaceutical grade Schisandra chinensis extract manufactured in China is standardized for a Schisandrol A content of no less than 2.0 weight percent. The pharmaceutical grade extract must pass extensive safety and efficacy procedures. Preferably, when employed in the practice of the present invention the Schisandra chinensis extract has a Schisandrol A content of from 1.0 to 50 weight percentage. Preferably, the Schisandra chinensis extract used in the present invention has a minimum Schisandrol A content of at least 2.0 weight percent.

The species of the present invention each support healthy liver function and in combination may be used to treat liver inflammation and the other conditions claimed.

According to a second aspect of the present invention there is provided. A method of treating a patient to alleviate or prevent one or more of the following:
i) Liver inflammation associated with hepatitis B virus;
ii) Liver inflammation associated with alcohol abuse;
iii) Metabolic disorders associated with the liver, including for example, diabetes and metabolic syndrome X;
iv) Fatty liver;
v) Treating patients who are non responsive to immuno-modulatory/antiviral combination therapies such as, interferon/ribovarin;
vi) As an adjunct therapy to combination therapies such as, interferon/ribovarin
vii) HCV associated liver disease;
viii) Hepatitis; fibrosis, cirrhosis or hepatocellular carcinoma;
ix) Treatment to reduce raised liver enzyme levels associated with chemotherapy
   comprising administering to the patient a composition consisting essentially of botanical raw materials, botanical drug substances or botanical ingredients from each of:
(a) The fruit of Silybum marianum;
(b) The root of Astragalus membranaceus var mongholicus or Hedysarum polybotrys;
(c) The root of Salvia miltiorrhiza, Salvia bowleyana or Salvia przewalskii; and
(d) The fruit of Schisandra chinensis or Schisandra sphenanthera

The botanical drug or dietary supplement preferably contains each species in an amount, relative to the total weight of all of the botanical raw materials or botanical ingredients, as follows:
(a) Silybum spp. from 22-48%;
(b) Astragalus spp. or Hedysarum spp. from 20-63%;
(c) Salvia spp. from 13-48%; and
(d) Schisandra spp. from 2-19%.

More preferably still each species is present in an amount as follows:
(a) Silybum spp. from 30-40%;
(b) Astragalu spp. or Hedysarum spp. from 20-30%;
(c) Salvias pp. from 20-30%; and
(d) Schisandras pp. from 7.5-15%.

Most preferably each species is present in the amounts as follows:
(a) Silybum spp. no less than 22% and more preferably no less than 30%;
(b) Astragalus spp. or Hedysarum spp. no less than 20%
(c) Salvia spp. no less than 13% and more preferably no less than 20%; and
(d) Schisandra spp. no less than 2% and more preferably no less than 7.5%.

According to the present invention, a therapeutically effective amount of the compositions of the invention are amounts sufficient to provide the claimed benefits while minimizing harmful side effects. In one embodiment, the therapeutically effective amount is an amount sufficient to reduce or alleviate the symptoms of liver inflammation without causing harmful side effects.

The dosage to be administered will vary and depend on the age, weight, sex and condition of the patient. Typical daily dosages of each of the plant based components (illustrated by way of example only with reference to the preferred species) are as follows (weights refer to a dry botanical raw material equivalent):

| | |
|---|---|
| Silybum marianum: | 2-15 g |
| Astragalus membranaceus var mongholicus: | 9-30 g |
| Salvia miltiorrhiza: | 9-15 g |
| Schisandra chinensis: | 1.5 g-6 g |

Dosages can be readily determined by one of ordinary skill in the art and can be readily formulated into the present supplemental and pharmaceutical compositions.

Botanical raw materials, botanical drug substances and botanical ingredients can be formulated into a medicament, dietary supplement or nutraceutical by conventional methods.

A nutraceutical is a food ingredient, food supplement or food product which is considered to provide a medical or health benefit, including the prevention and treatment of disease. In general a nutraceutical is specifically adapted to confer a particular health benefit on the consumer. A nutraceutical typically comprises a micronutrient such as a vitamin, mineral, herb or phytochemical at a higher level than would be found in a corresponding regular food product. That level is typically selected to optimise the intended health benefit of the nutraceutical when taken either as a single serving or as part of a diet regimen or course of nutritional therapy.

A botanical drug or dietary supplement of the present invention may be formulated into a medicament or dietary supplement by mixing with a dietetically or pharmaceutically acceptable carrier or excipient. Such a carrier or excipient may be a solvent, dispersion medium, coating, isotonic or absorption delaying agent, sweetener or the like. Suitable carriers may be prepared from a wide range of materials including, but not limited to, diluents, binders and adhesives, lubricants, disintegrants, colouring agents, bulking agents, flavouring agents, sweetening agents and miscellaneous materials such as buffers and adsorbents that may be needed in order to prepare a particular dosage form. The use of such media and agents for pharmaceutically active substances is well known in the art. Except insofar as any conventional media or agent is known to be incompatible with the plant based composition of the present invention, its use in the present compositions is contemplated.

For example, a solid oral forms may contain, together with the active components, diluents such as lactose, dextrose, saccharose, cellulose, corn starch or potato starch; lubricants such as silica, talc, stearic acid, magnesium or calcium stearate and/or polyethylene glycols; binding agents such as starches, arabic gums, gelatin, methylcellulose, carboxymethylcellulose, or polyvinyl pyrrolidone; disintegrating agents such as starch, alginic acid, alginates or sodium starch glycolate; effervescing mixtures; dyestuffs, sweeteners; wetting agents such as lecithin, polysorbates, lauryl sulphates and macrogol polyethylene glycol). Such preparations may be manufactured in known manners, for example by means of mixing, granulating, tabletting, sugar coating, or film-coating processes.

Liquid dispersions for oral administration may include water solutions, tinctures, syrups, emulsions and suspensions. The syrups may contain as carrier, for example, saccharose or saccharose with glycerol and/or mannitol and/or sorbitol. In particular, a syrup for diabetic patients can contain as carriers only products, for example sorbitol, which do not metabolise to glucose or which only metabolise a very small amount to glucose. The suspensions and the emulsions may contain as carrier, for example, a natural gum, agar, sodium alginate, pectin, methylcellulose, carboxymethylcellulose or polyvinyl alcohol.

The botanical drug or dietary supplement of the present invention is also suitably formulated into granules or a powder. In this form it can be readily dispersed in water or other liquid such as tea or a soft drink for human patients to drink. It may also be encapsulated, tabletted or formulated with a physiologically acceptable vehicle into unit dosage forms. A unit dosage can comprise a therapeutically effective amount of the extract for a single daily administration, or it can be formulated into smaller quantities to provide for multiple doses in a day. The composition may thus, for instance, be formulated into tablets, capsules, syrups, elixirs, enteral formulations or any other orally administrable form. Examples of physiologically acceptable carriers include water, oil, emulsions, alcohol or any other suitable material

The present invention will be further illustrated, by way of Example, only with reference to the following formulations and data in which:

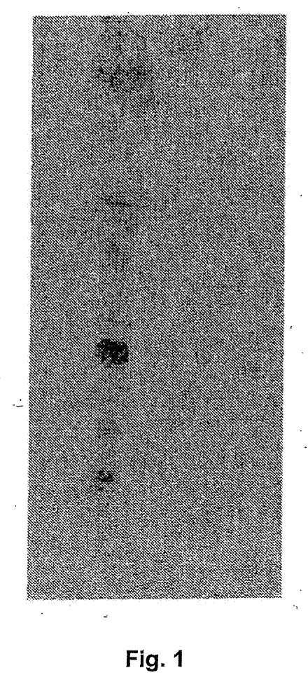

FIG. 1 is a TCL picture of the BDS of Astragalus membranaceus var mongholicus;

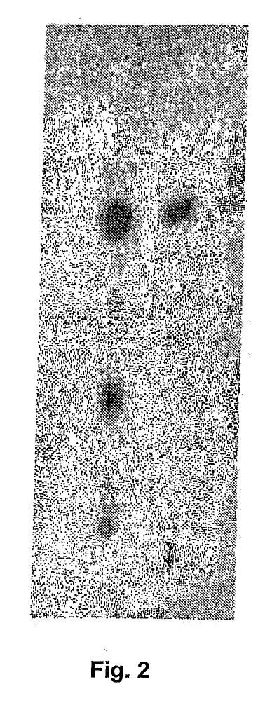

FIG. 2 is a TCL picture of the BDS of Salvia miltiorrhiza;

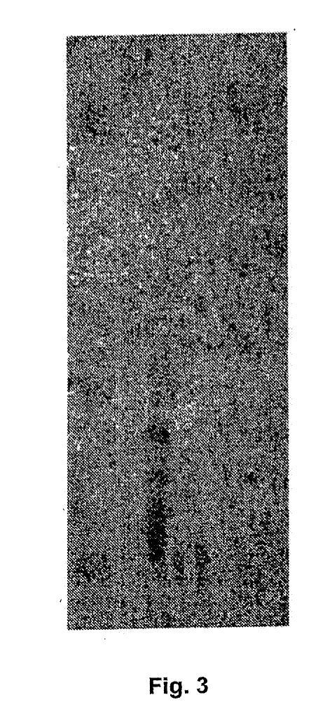

FIG. 3 is a TCL picture of the BDS of Schisandra chinensis.

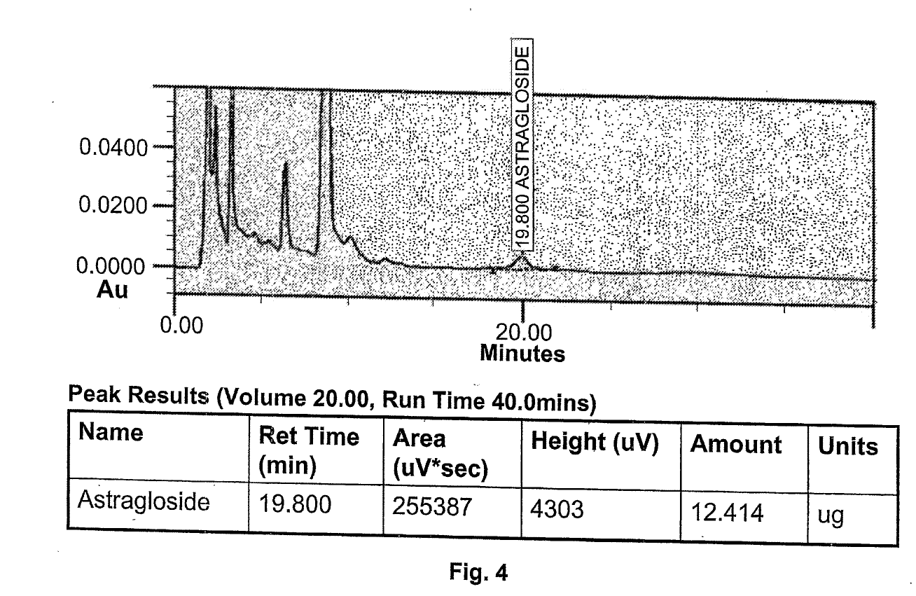

FIG. 4 is a HPLC chromatogram of the BDS of Astragalus membranaceus;

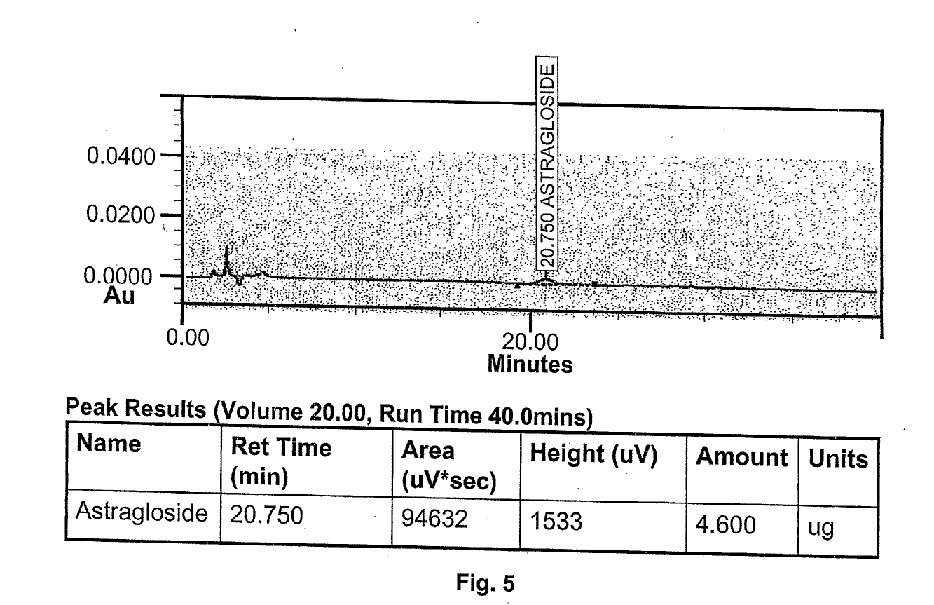

FIG. 5 is a HPLC chromatogram of Astragaloside (a marker of Astragalus membranaceus var mongholicus)

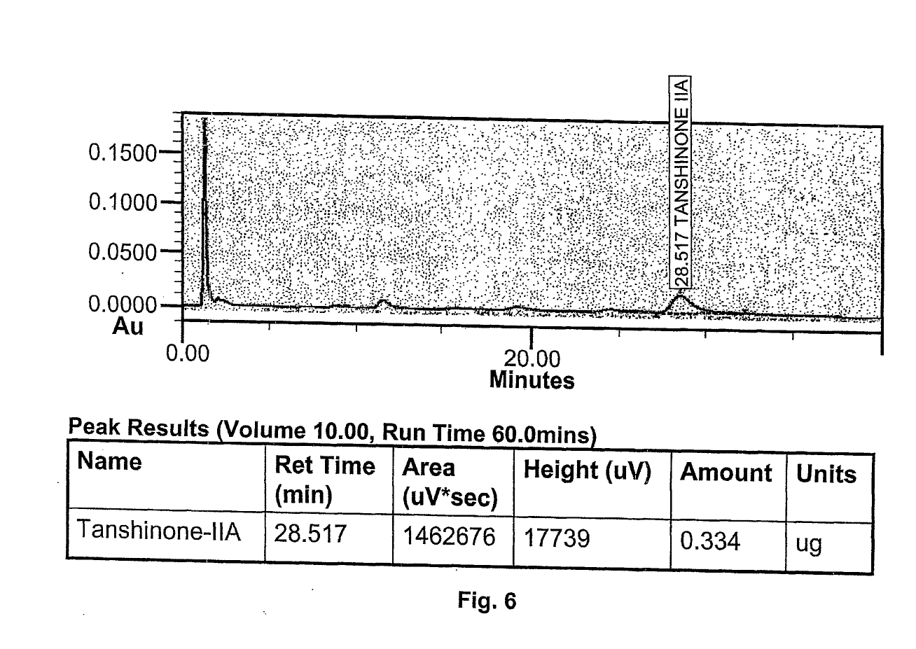

FIG. 6 is a HPLC chromatogram of the BDS of Salvia miltiorrhiza;

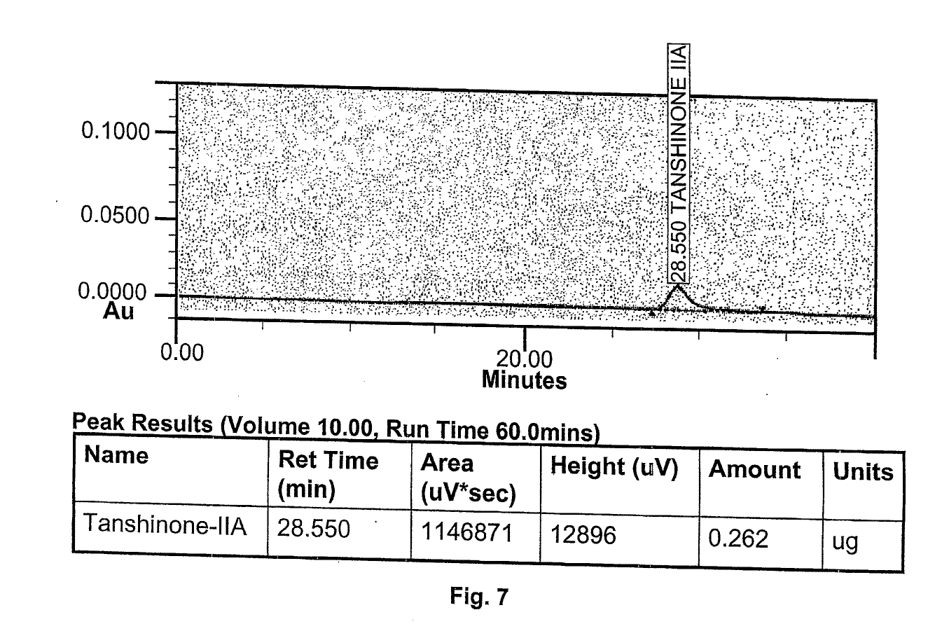

FIG. 7 is a HPLC chromatogram of Tanoshone-JIA (a marker of Salvia miltiorrhiza)

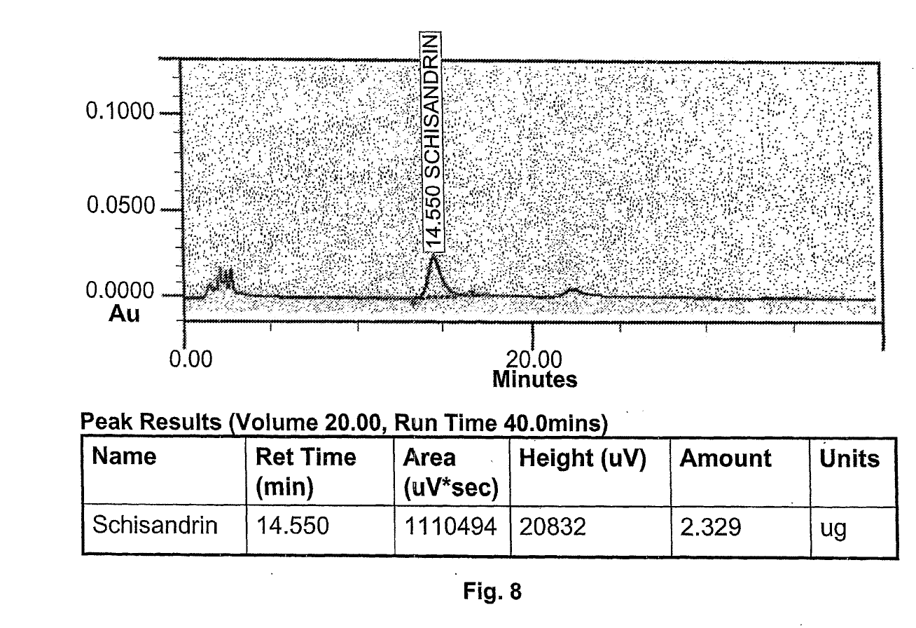

FIG. 8 is a HPLC chromatogram of the BDS of Schisandra chinensis;

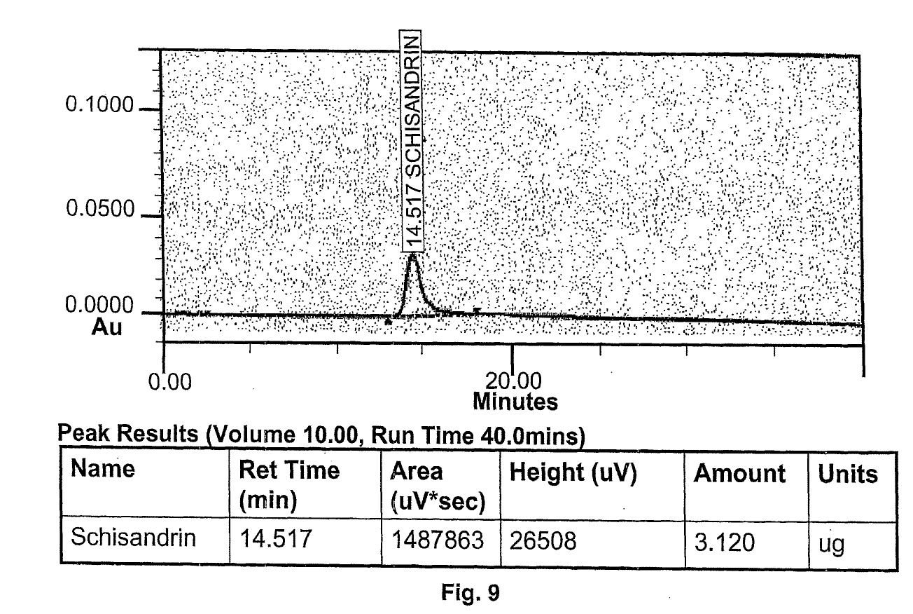

FIG. 9 is a HPLC chromatogram of Schisandrin (a marker of Schisandra chinensis);

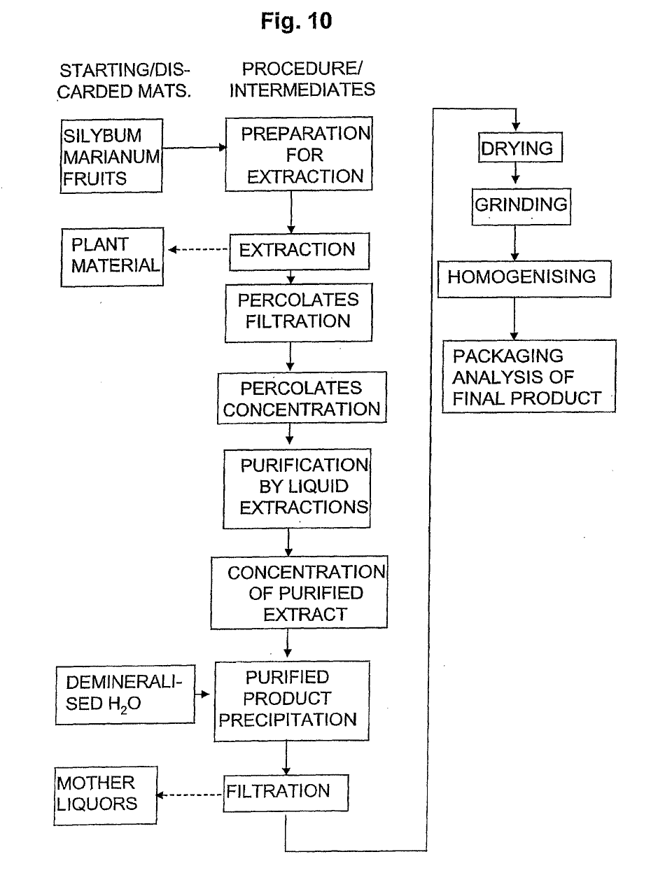

FIG. 10 is a flow chart showing the manufacture process for producing a botanical drug substance from Silybum spp.;

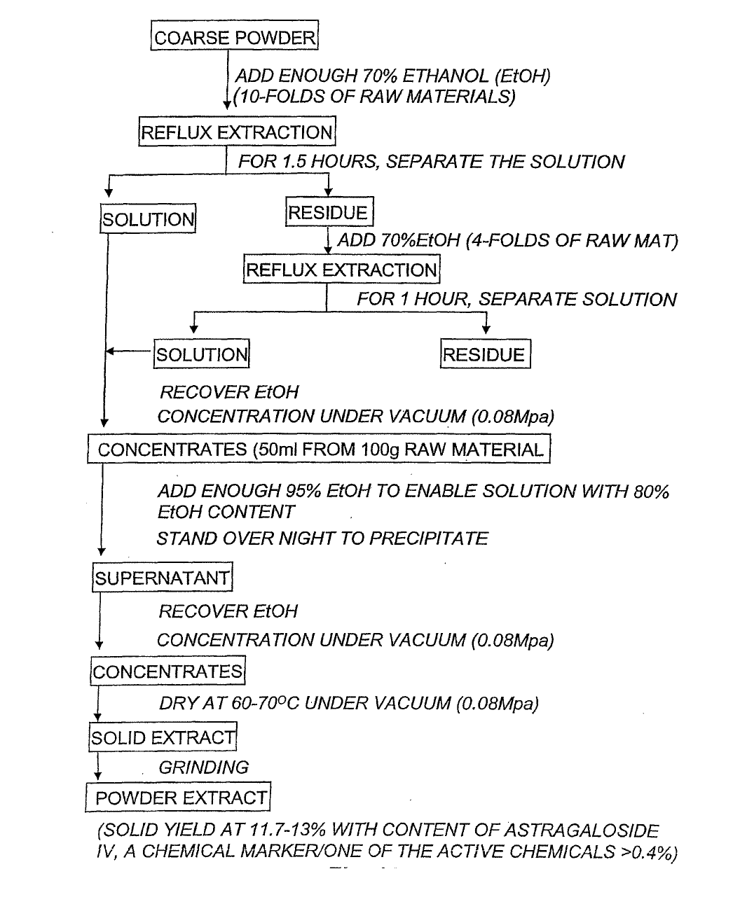

FIG. 11 is a flow chart showing the manufacture process for producing a botanical drug substance from Astragalus spp.;

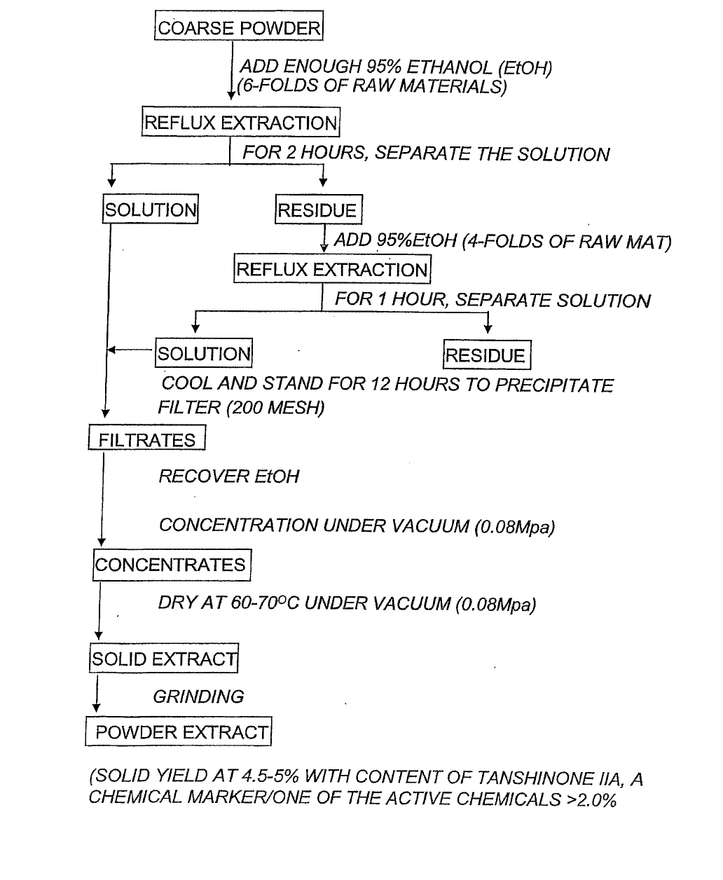

FIG. 12 is a flow chart showing the manufacture process for producing a botanical drug substance from Salvia spp.;

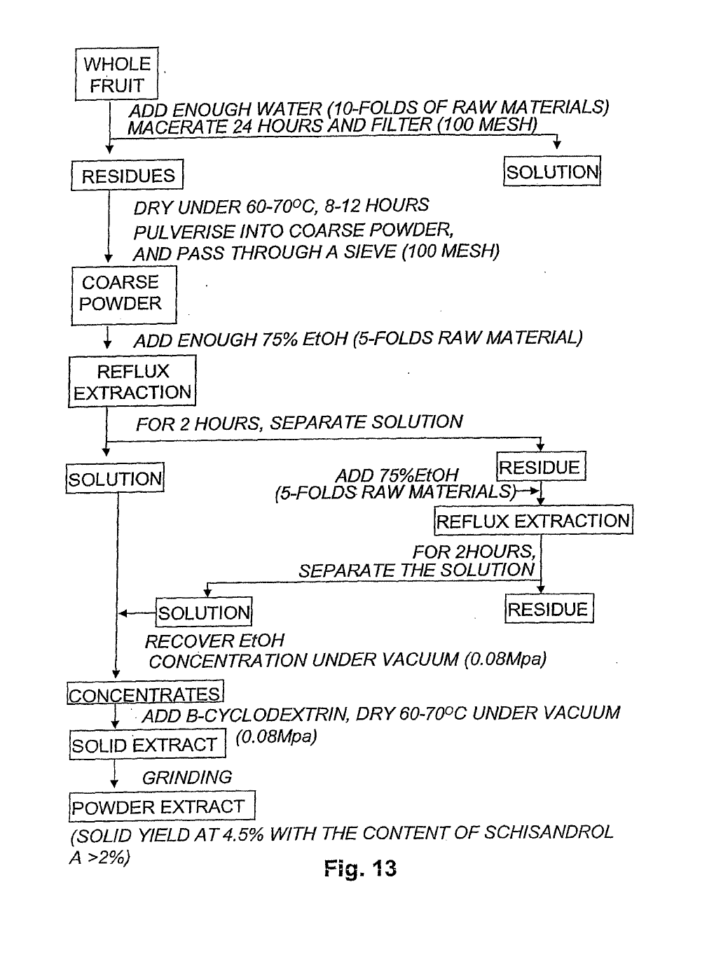

FIG. 13 is a flow chart showing the manufacture process for producing a botanical drug substance from Schisandra spp.

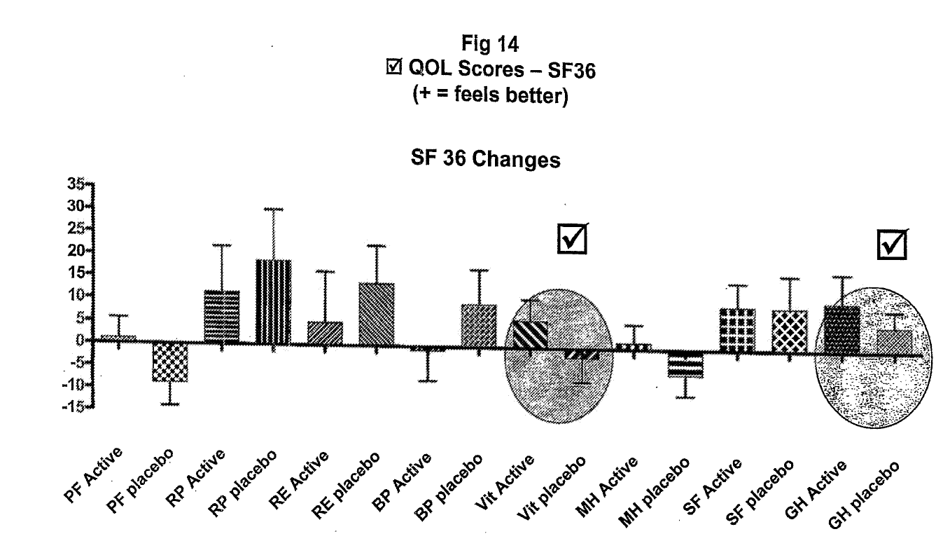

FIG. 14 shows the quality of life scores (SF36) from a clinical trial on hepatitis C patients;

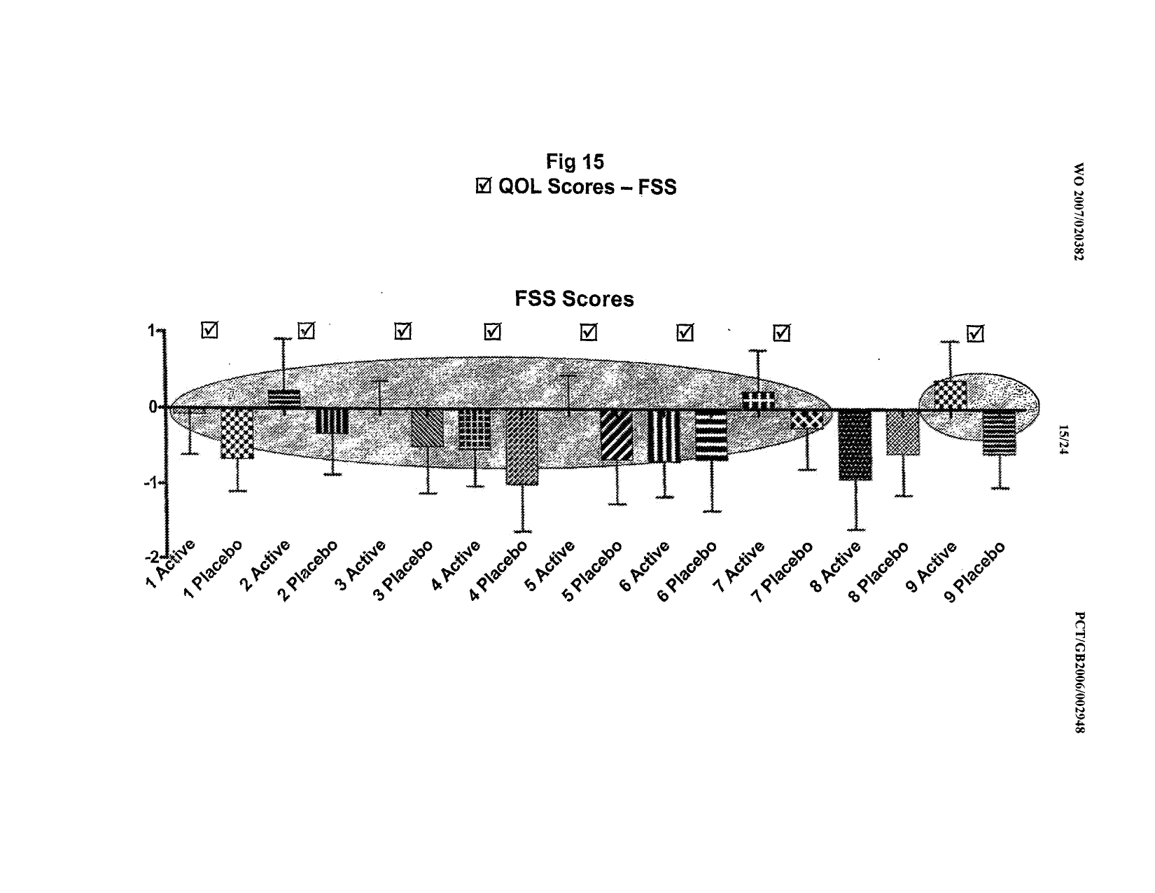

FIG. 15 shows the quality of life scores (FSS) from a clinical trial on hepatitis C patients;

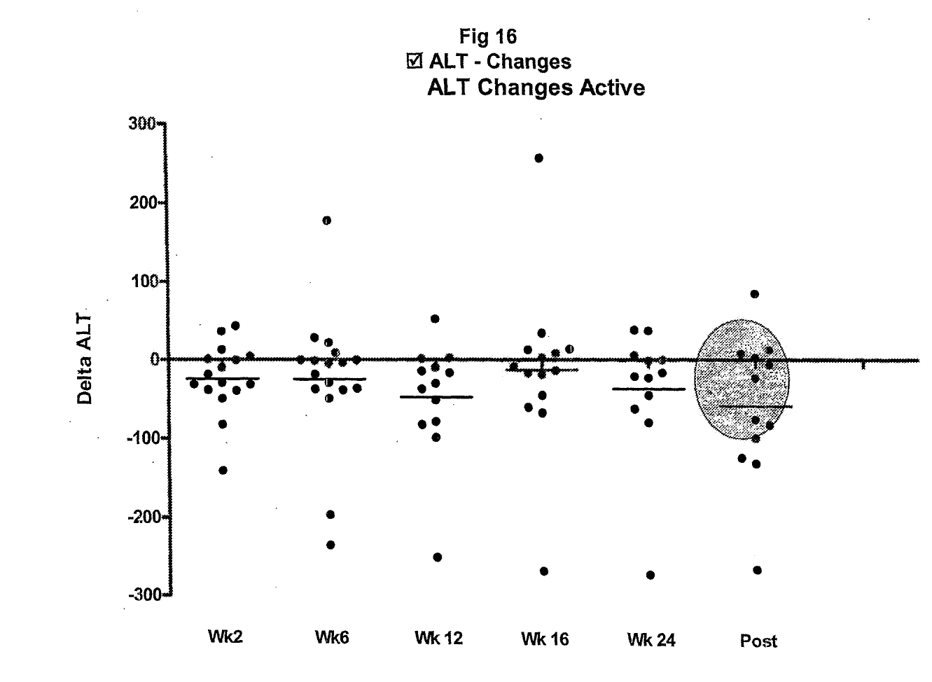

FIG. 16 shows the changes in ALT enzyme levels in patients taking the “active” in the clinical trial

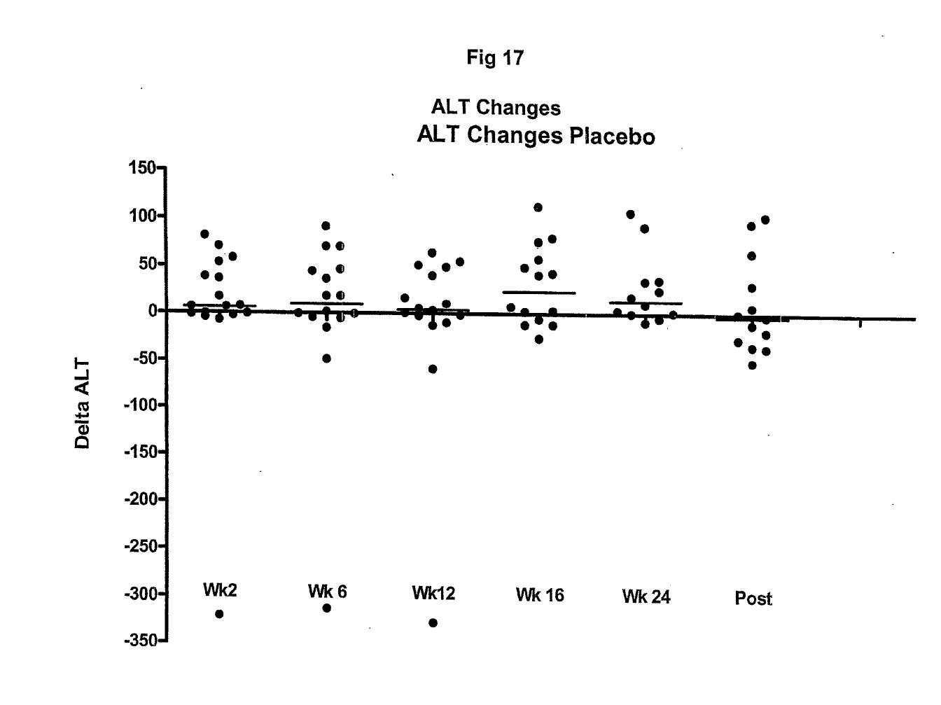

FIG. 17 Shows the changes in ALT enzyme levels in patients taking the “placebo” in the clinical trial

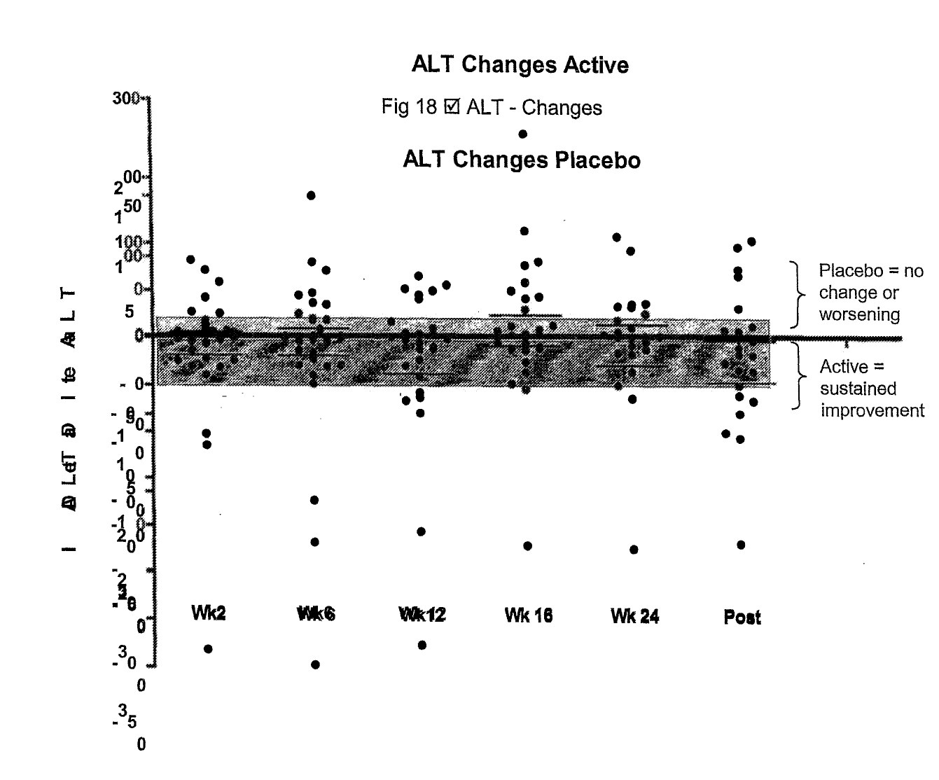

FIG. 18 shows a comparison of the FIGS. 16 and 17 data superimposed for comparability

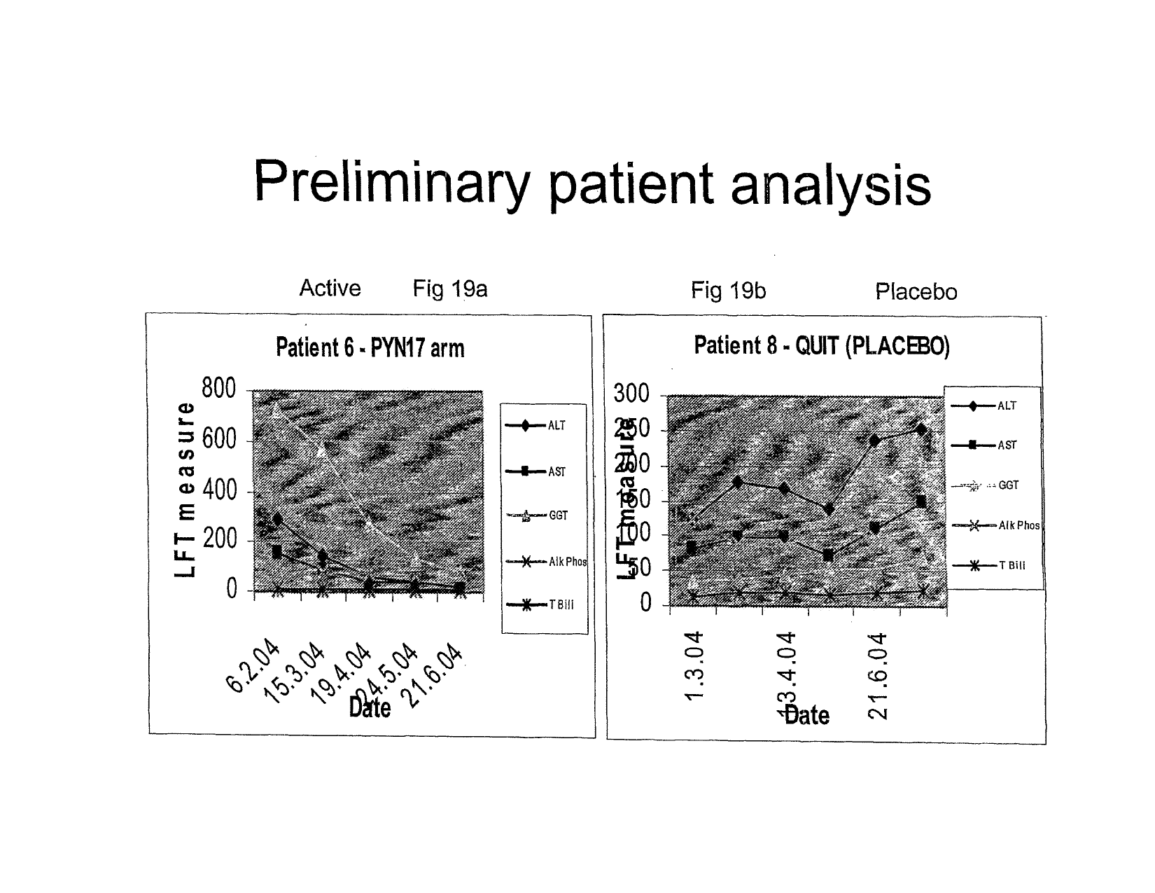

FIG. 19 a (active) and b (placebo) show a comparison of various enzymes which give a secondary measure of liver inflammation

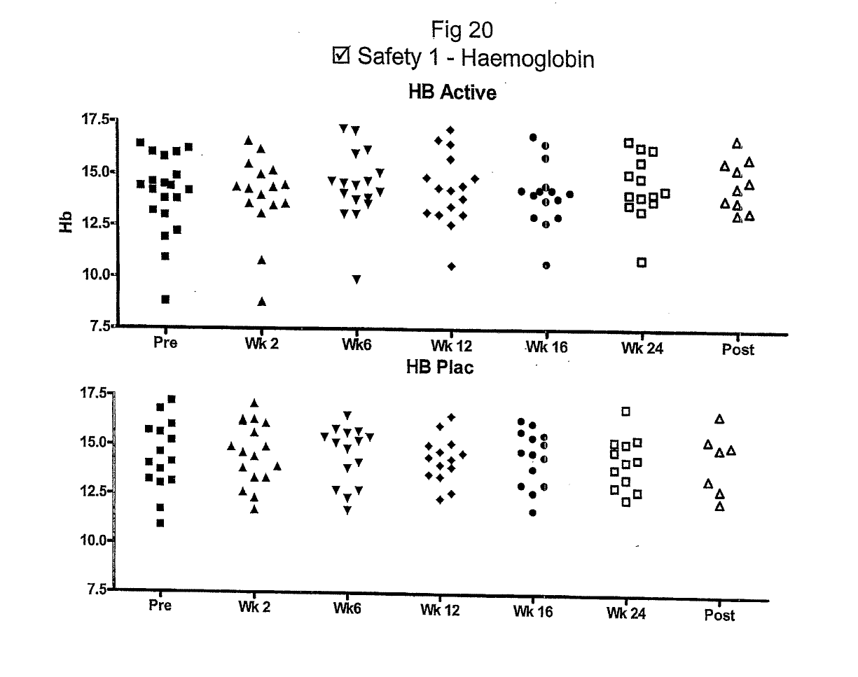

FIG. 20 illustrated haemoglobin safety data

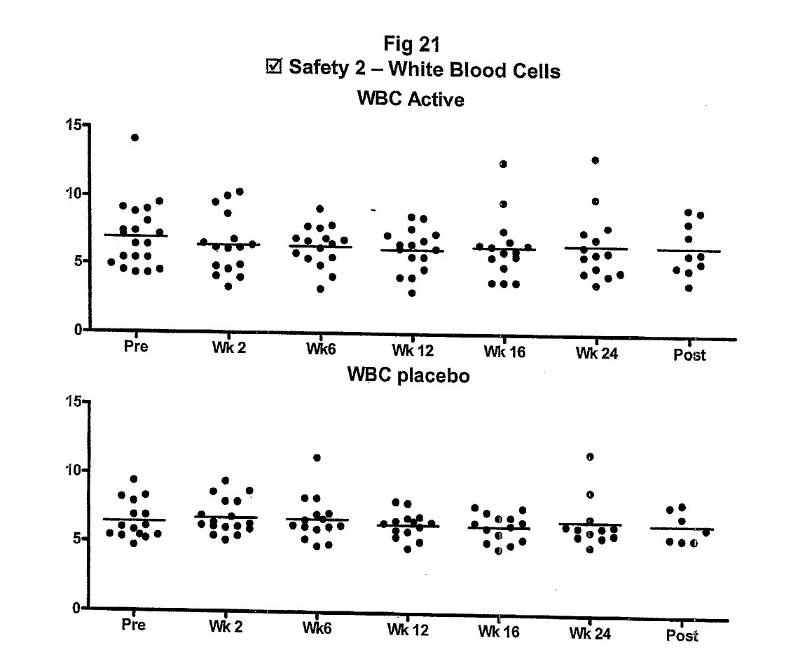

FIG. 21 illustrates white blood cell safety data

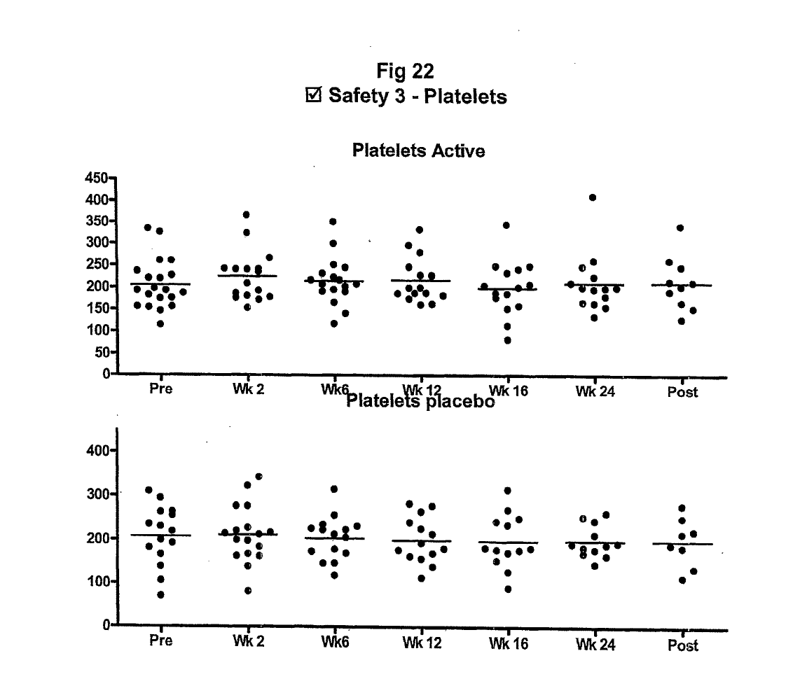

FIG. 22 illustrates platelet safety data

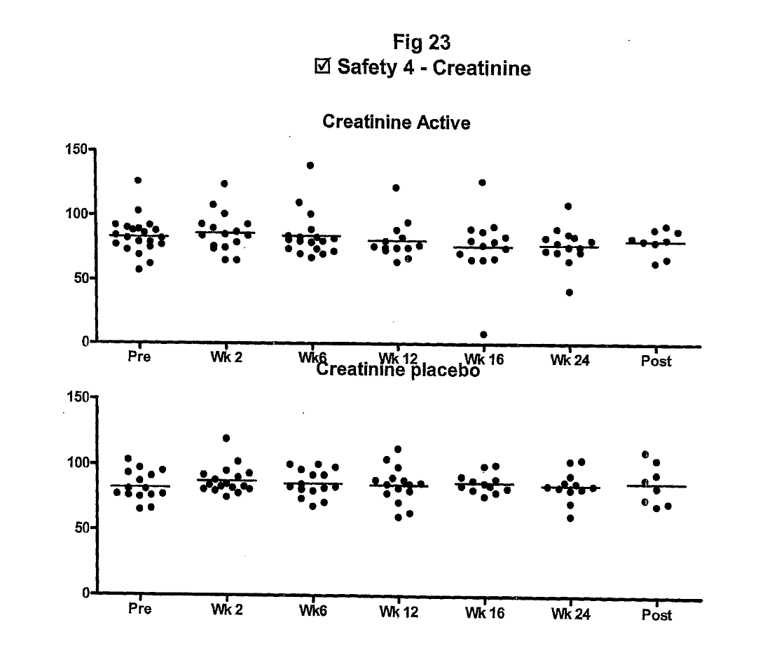

FIG. 23 illustrates creatinine safety data and

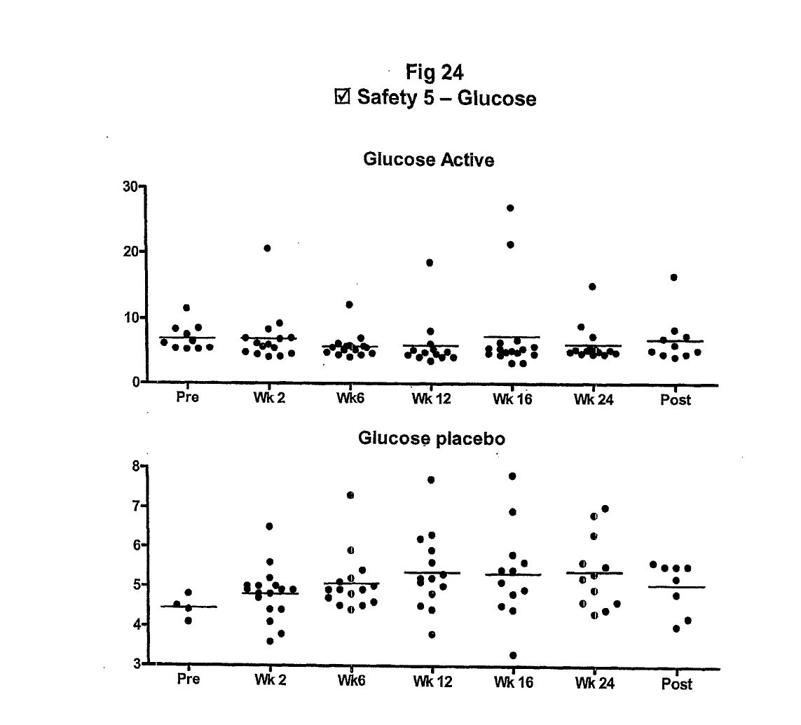

FIG. 24 illustrates glucose safety data.

### DETAILED DESCRIPTION

### Example 1

### Preparation of Botanical Drug from Botanical Drug Substances

Standardised extracts of Silybum marianum (fruit), Salvia miltirrhiza (root), Schisandra chinensis (fruit), and Astragalus membranaceus var mongholicus (root) were made separately using extraction procedures designed specifically for each herb in order to achieve the desired therapeutic potency of the extracts. The extracts were dried and the resulting dry powdered extracts mixed in the proportions shown below (the weights are given both for the extracts and as an equivalent by weight of dry botanical raw material).
(a) Silybum marianum; from 0.200 g to 0.250 g (equivalent to 12 g to 15 g of botanical raw material),
(b) Astragalus membranaceus var mongholicus; 0.585 g to 1.95 g (equivalent to 9 g to 30 g of botanical raw material)
(c) Salvia miltirrhiza; 0.225 g to 0.375 g (equivalent to 9 g to 15 g f botanical raw material) and
(d) Schisandra chinensis; 0.150 g to 0.600 g (equivalent to 1.5 g to 6 g of botanical raw material).

### Example 2

### Formulation into a Suspension Mixture

The spray-dried botanical drug substances of Example 1 were formulated into a suspension dosage form by mixing the spray-dried botanical drug substances with:
a) one or more gellants or thickeners comprising at least one xanthum gum having a particle size distribution such that 100% by weight of the particles pass a 60 mesh sieve, 95% by weight of the particles pass a 80 mesh sieve and 70% by weight of the particles pass a 200 mesh sieve,
b) one or more fillers; and
c) one or more wetting agents and or surfactants.

The resulting formulation, referred to as the PYN17 suspension powder mixture, contained the following:

Composition: per sachet

| Composition: | per sachet |
|---|---|
| Active ingredients: | |
| Milk Thistle Fruit dry extract | 0.200 g |
| Chinese Sage Root dry extract | 0.225 g |
| Schisandra Fruit dry extract | 0.400 g |
| Astralagus Root dry extract | 0.585 g |
| Excipients: | |
| Macrogol 6000 powder | 0.600 g |
| Ferwogel 30.385 (molecular weight | 0.070 g |
| 3.5-4.0 × 10⁶) | |
| Mannitol EZ | 0.160 g |
| Aerosil 200 | 0.050 g |
| Aspartame | 0.050 g |
| Caramel powder | 0.100 g |
| Peppermint powder aroma | 0.060 g |

### Example 3

### Activity of PYN17 Suspension Powder Mixture

A sachet of the suspension powder was re-suspended in 2.5 ml water and further diluted 1 in 7. The incompletely dissolved suspension was filtered and the soluble fraction tested.

10 μl of solution was tested in 100 μl culture of cells at a concentration of 1/70. Concentrations of 1/350 and 1/1750 were also used to determine toxicity.

To test toxicity the cells were cultured with Replicon cells for 72 hours, and tritiated thymidine was added 18 hours prior to harvesting.

Results:

Tritiated thymidine incorporation

| Dilution | Well 1 | Well 2 | Well 3 | Well 4 | Well 5 | Mean |
|---|---|---|---|---|---|---|
| PYN-17 | cpm | cpm | cpm | cpm | cpm | cpm |
| 1/70 | 18 | 24 | 65 | 51 | 77 | |
| 1/350 | 41010 | 32432 | 34719 | 30311 | 32371 | 34169 |
| 1/1750 | 36210 | 28315 | 32424 | 38230 | 39815 | 34999 |
| 0 | 31609 | 35373 | 36199 | 36281 | 36210 | 35134 |

Inhibition of replication measured by expression of Renilla luciferase.

The 1/70 dilution was toxic to the cells (as under the microscope the cells were dead). This dilution was not used in the Replicon assay and a further lower dilution was used.

| | Well 1 | | | | | | |
|---|---|---|---|---|---|---|---|
| Dilution | luciferase | | | | | Mean luciferase | |
| PYN17 | activity | Well 2 | Well 3 | Well 4 | Well 5 | activity | +/−SD |
| 1/350 | 531292 | 234958 | 614669 | 479425 | 725350 | 517139 | 183108 |
| 1/1750 | 594920 | 972891 | 889324 | 595922 | — | 763264 | 196789 |
| 1/8750 | 880338 | 1005370 | 608077 | 644105 | 806756 | 788929 | 165228 |
| 0 | 1139829 | 870757 | 820645 | 724027 | — | 888815 | 178079 |

Conclusion

At a 1/350 dilution an inhibition of 41.8% was noted indicating activity against Hepatitis C virus.

The results may be slightly skewed by one very low result (well 2).

The control (no suspension powder) may also be skewed by the one high result (well 1).

At 1/350 the mean without the low result was 587684

The control without the high result (well 1) was 805143

Excluding the single high and low results the % inhibition was 27%.

### Examples 4-7

These illustrate the extraction methods used in the preparation of the botanical drug substances used in the botanical drug of the invention.

### Example 4

### Preparation of a Botanical Drug Substance from a Silybum spp

Referring to FIG. 10 there is illustrated a process for producing a botanical drug substance of a Silybum spp. The fruits are prepared for extraction, undergo an extraction, the resulting solution is filtered, and concentrated. The concentrated purified extract then undergoes a further clean up process in which purified product is precipitated, filtered and the filtrate dried and ground for packing. Such a product can be obtained from Indena SpA.

### Example 5

### Preparation of a Botanical Drug Substance from a Astragalus spp

(The preparation of a botanical drug substance from a Hedysarum spp. is equivalent) Referring to FIG. 11 Astragalus spp. root material is dried in an oven at 60° C. for 3 hours, pulverised into a coarse powder, passed through a sieve (10 mesh) and subjected to extraction as per the flow chart. The extraction process is an ethanolic extraction. The concentrate obtained is re-dissolved in ethanol, any precipitate removed and the product concentrated and dried. The method yields a solid content in excess of 10% with an Astragaloside content of greater than 0.4%.

### Example 6

### Preparation of a Botanical Drug Substance from a Salvia spp

Referring to FIG. 12 the Salvia spp. root material is dried in an oven at 60° C. for 3 hours, pulverised into a coarse powder, passed through a sieve (10 mesh) and subjected to extraction as per the flow chart. The extraction process is an ethanolic extraction and the resulting concentrate is dried. The method yields a solid content in excess of 4% with a Tanshinone IIA content of greater than 1.5%.

### Example 7

### Preparation of a Botanical Drug Substance from a Schisandra spp

Referring to FIG. 13 the Salvia spp. fruit is macerated in water and filtered. The filtrate residues are dried, powdered and subjected to an ethanolic extraction, and the resulting concentrate is dried. The method yields a solid content in excess of 4% with a Schisandrol A content of greater than 2%.

### Examples 8-11

A botanical drug substance obtained from the sources identified, and by the methods described was subject to analysis and the results are given below:

### Example 8

The botanical drug substance from a Silybum spp. was shown by analysis to have the following characteristics

| DETERMINATION | RESULTS | SPECIFICATIONS | U.M |
|---|---|---|---|
| SPECTROPHOTOMETRIC CONTENTS | 70.9 | >=65.0 | % |
| of silymarin, calculated as silybin, according to DAB10 | | | |
| HPLC CONTENTS | 38.8 | >=30.0 | % |
| As sum of silybin and isosilybin | | | |
| CHARACTERS | Complies | Complies | |
| Brownish yellow powder | | | |
| SOLUBLE SUBSTANCES | 0.25 | <=0.5 | % |
| in pantane | | | |
| HPLC: IDENTIFICATION LOSS ON DRYING | Complies | Complies | |
| (T = 80° C., in vacuum t = 3 h | 0.0 | <=5.0 | % |
| SULPHATED ASH | 0.33 | <=1.0 | % |
| According to Ph. Eur. | | | |
| HEAVY METALS | Complies | <=100 | ppm |
| According to Ph. Eur. Method A | | | |
| RESIDUAL ORGANIC SOLVENTS | | | |
| Ethanol | 0.4 | <=1.0 | % |
| Ethyl Acetate | <0.0008 | <=0.01 | % |
| Hexane | Complies | <=0.01 | % |
| MICRBIOLOGICAL CONTROL | | | |
| According to Ph. Eur. | | | |
| BACTERIA | <1000.0 | <=1000.0 | ofu/g |
| Maximum limit of acceptance: | | | |
| 5 × 1000 cfu/g | | | |
| TM/0113 | | | |
| FUNGI | <100.0 | <=100.0 | cfu/g |
| Maximum limit of acceptance: | | | |
| 5 × 100 cfu/g | | | |
| TM/0118 | | | |
| ENTEROBACTERIA | <100.0 | <=100.0 | cfu/g |
| TM/0015 and TM/0075 | | | |
| STAPHYLOCOCCUS AUREUS. SALMONELLA | Absent | Absent | |
| TM/0008, TM/0009, TM/0017 and TM/0075 | | | |
| ESCHERICHIA COLI, PSEUDOMONAS | Absent | Absent | |
| AERUGINOSA | | | |
| TM/0010, TM0011, TM0016 and TM/0075 | | | |

### Example 9

The botanical drug substance from the Astragalus spp. was shown by analysis to have the following characteristics:

A) Certificate of Analysis

Product Name: Astragalus Root Extract (Astragalus membranaceus var mongholicus)

Batch Number: AMR-200201PE

| TESTS | | SPECIFICATION | RESULT |
|---|---|---|---|
| Appearance | | Pale yellow colour | Pass |
| Loss on Drying: | | <5% (CP) | 2.65% |
| Particle Size: | | 80 mesh | Pass |
| Total Ash | | <5.0% | 0.14% |
| Heavy Metals: | Lead | <5 ppm | 0.55 |
| | Mercury | <1 ppm | 0.84 |
| | Arsenic | <1 ppm | 0.61 |
| | Cadmium | <0.5 ppm | 0.21 |

| | | |
|---|---|---|
| Acid Insoluble Ash | <2.0% | 0.026% |
| Microbial Total viable aerobic | <10³ cfu/g | 80 |
| count: | | |
| Fungal & Yeast: | <10² cfu/g | 10 |
| Escherichia coli: | Absent in 10 g | Absent |
| Salmonella spp.: | Absent in 10 g | Absent |
| Content Assay: | Astragaloside IV >0.4% | 0.44% |

B) Chemical Analysis

Name of the Product: Astragalus Root Extract (Astragalus membranaceus var mongholicus)

Batch Number: AMR-200201PE

Chemical Analysis:

i) TLC Fingerprint: See FIG. 1 which is a TLC picture of the BDS of Astragalus membranaceus var mongholicus. The left is the BDS sample and the right the standard reference chemical Astragaloside IV

Preparation of Test Solutions:

Add 40 ml of methanol to 1 g of powder extract, shake well and filter. Apply the filtrates to a prepared neutral aluminium oxide column, then follow the method described in Chinese Pharmacopoeia (English Edition, 2000), Page 161, Identification (2),

Reference solution: Dissolve chemical reference standard (CRS) Astragaloside IV in methanol to produce a 1 mg/l ml reference solution.

Loadings: Load 2 μl of the test solution and 2 μl of the reference solution, respectively, on foil-backed Silica gel F₂₅₄ plate (Merck).

Developing solvent system: chloroform:methanol:water (13:7:2) (Lower layer)

Developing: Add mixed developing solution to a TLC tank and stand for 15 Minute for equilibrium. Put the TLC plate in and develop for 7.5 cm.

Detection:

When sprayed with 10% of sulphuric acid in ethanol and heated at 105° C. a brown spot is obtained in TLC chromatogram of the test solution corresponds in position and colour to the spot of the reference solution. Observe the developed TLC plate under UV365ₙₘ light, both reference chemical Astragaloside IV and test solution showed an orange yellow spot at Rf 0.49,

ii) HPLC analysis

Equipment: Waters HPLC System, LC 600 pump and UV detector (Model 486).

Column: Spherisorb S100Ds1, 25 cm×4.6 mm

Column temperature: 25° C.

Flow rate: 1.0 ml/min

Detection wavelength: UV200ₙₘ

Mobile phase: acetonitrile:water (1:2)

Preparation of CRS solution: Dissolve 2 mg of Astragaloside IV in mobile phase solution in a 10 ml volumetric flask.

Preparation of Test Solutions:

Weigh accurately 1.0 g of powder extract, add 50 ml of 2% KOH in methanol, heat and reflux on water bath for 1 hour and filter. Repeat the procedure for three times. Combine the filtrates and recover the solvent. Add 25 ml of water to dissolve the residue, wash with 50 ml of ether. To the aqueous solution, extract with 25 ml of n-butanol (saturated in water) for three times. Combine butanol solution, wash twice with 25 ml of water, respectively, then wash with 25 ml of potassium dihydrogen phosphate, recover the solvent. Add accurately 10 ml of mobile phase solution to the residue shake well, filter through Millipore (0.45 μm) as test solution.

Quantity of injection: Inject 20 μl of CRS solution and 20 μl of test solution, respectively.

Result: See chromatograms in FIGS. 4 and 5. FIG. 4 (the BDS) shows at least 10 clearly identifiable peaks including Astragaloside IV at a retention time of about 20 minutes. The area under the graph indicates a presence of at least 0.4% by weight of Astragaloside IV. The FIG. 5 chromatogram is a control with the marker alone.

| Specifications for Astragaloside IV content | |
|---|---|
| (% w/w) | Result (% w/w) |
| >0.4 | 0.44 |

### Example 10

The botanical drug substance from the Salvia spp. was shown by analysis to have the following characteristics:

A) Certificate of Analysis

Product Name Salvia Miltiorrhiza Root Extract (salvia miltiorrhiza)

Batch Number: SMR-200201PE

| TESTS | | SPECIFICATION | RESULT |
|---|---|---|---|
| Appearance | | Dark red colour | Pass |
| Loss on Drying: | | <5% (CP) | 3.24% |
| Particle Size: | | 80 mesh | Pass |
| Total Ash | | <5.0% | 0.38% |
| Acid Insoluble Ash | | <2.0% | 0.04% |
| Heavy Metals: | Lead | <5 ppm | 0.65 |
| | Mercury | <1 ppm | 0.14 |
| | Arsenic | <1 ppm | 0.62 |
| | Cadmium | <0.5 ppm | 0.38 |

| | | |
|---|---|---|
| Microbial Total viable aerobic | <10³ cfu/g | 100 |
| count: | | |
| Fungal & Yeast: | <10² cfu/g | 20 |
| Escherichia coli: | Absent in 10 g | Absent |
| Salmonella spp.: | Absent in 10 g | Absent |
| Content Assay: | Tanshinone□_{A} > 1.5% | 1.98% |

B) Chemical Analysis

Name of the Product: Salvia Miltiorrhiza Root Extract (Salvia miltiorrhiza)

Batch Number: SMR-200201PE

Chemical Analysis:

i) TLC Fingerprints: See FIG. 2 which is a TLC picture of the BDS of Salvia miltiorrhiza. The left is the BDS sample and the right the standard reference chemical Tanshinone IIA

Preparation of Test solutions: Add 1 ml of ethyl acetate to 100 mg of powder extract

Reference solution: Dissolve chemical reference standard (CRS) Tanshinone II_{A} in ethyl acetate to produce a 2 mg/1 ml reference solution.

Loadings: Load 5 μl of the test solution and 5 μl of the reference solution, respectively, on foil-backed Silica gel plate (Merck).

Developing solvent system: benzene: ethyl acetate (19:1)

Developing: Add mixed developing solution to a TLC tank and stand for 15 Minute for equilibrium. Put the TLC plate in and develop for 7.5 cm.

Detection: Dry the developed plate in air, a dark red spot obtained in TLC chromatogram of the test solution corresponds in position and colour to the spot of the reference solution at Rf 0.46.

ii) HPLC analysis

Equipment: Waters HPLC System, LC 600 pump and UV detector (Model 486).

Column: Spherisorb S100Ds1, 25 cm×4.6 mm

Column temperature: 25° C.

Flow rate: 1.0 ml/min

Detection wavelength: UV270ₙₘ

Mobile phase: Methanol:Water (15:5)

Preparation of CRS solution: Weight accurately 10 mg of Tanshinone IIA to a 50 ml amber volumetric flask and dissolve with methanol to the volume. Accurately measure 2 ml to a 25 ml amber volumetric flask and add methanol to the volume.

Preparation of test solutions: Weigh accurately 30 mg of powder extract to a 25 ml volumetric flask, add 18 ml of methanol and treat under ultrasonic for 5 minutes, then add methanol to the volume.

Quantity of injection: Inject 5 μl of CRS solution and 5 μl of test solution, respectively.

Result: See chromatograms in FIGS. 6 and 7. FIG. 6 (the BDS) shows at least 6 identifiable peaks including Tanshinone IIA at a retention time of about 28/29 minutes. The area under the graph indicates a presence of at least 1.5% by weight of Tanshinone IIA. The FIG. 7 chromatogram is a control with the marker alone.

| Specifications for Tanshinone II_{A} content | |
|---|---|
| (% w/w) | Result (% w/w) |
| >1.5 | 1.98 |

### Example 11

The botanical drug substance from the Schisandra spp. was shown by analysis to have the following characteristics:

A) Certificate of Analysis

Product Name Schisandra Fruit Extract (Schisandra chinensis)

Batch Number: SCF-200201PE

| TESTS | | SPECIFICATION | RESULT |
|---|---|---|---|
| Appearance | | Brownish red colour | Pass |
| Loss on Drying: | | <5% (CP) | 4.5% |
| Particle Size: | | 80 mesh | Pass |
| Total Ash | | <5.0% | 0.25% |
| Acid Insoluble Ash | | <2.0% | 0.06% |
| Heavy Metals: | Lead | <5 ppm | 0.45 |
| | Mercury | <1 ppm | 0.47 |
| | Arsenic | <1 ppm | 0.74 |
| | Cadmium | <0.5 ppm | 0.36 |

| | | |
|---|---|---|
| Microbial Total viable aerobic | <10³ cfu/g | 90 |
| count: | | |
| Fungal & Yeast: | <10² cfu/g | 10 |
| Escherichia coli: | Absent in 10 g | Absent |
| Salmonella spp.: | Absent in 10 g | Absent |
| Content Assay: | Schizandrol A > 2.0% | 2.4% |

B) Chemical Analysis

Name of the Product: Schisandra Fruit Extract (Schisandra chinensis)

Batch Number: SCF-200201PE

Chemical Analysis:

i) TLC Fingerprints: See FIG. 3 which is a TLC picture of the BDS of Schisandra chinensis. The left is the BDS sample and the right the standard reference chemical Schisandrin A

Preparation of Test Solutions:

Add 20 ml of chloroform to 0.5 g of powder extract, ultrasonicate for 10 minutes and filter. Evaporate the filtrates to dryness and dissolve the residue in 1 ml of chloroform as test solution.

Reference solution: Dissolve chemical reference standard (CRS) Schizandrol A in chloroform to produce a 1 mg/1 ml reference solution.

Loadings: Load 2 μl of the test solution and 2 μl of the reference solution, respectively, on foil-backed Silica gel F₂₅₄ plate (Merck).

Developing solvent system: Petroleum ether (30-60° C.): ethyl formate: formic Acid (15:5:1) (upper layer)

Developing: Add mixed developing solution to a TLC tank and stand for 15 minute for equilibrium. Put the TLC plate in and develop for 7.5 cm.

Detection: Dry the developed plate in air, observe the plate under UV 254 nm, a dark spot obtained in TLC chromatogram of the test solution corresponds in position and colour to the spot of the reference solution at Rf 0.14.

ii) HPLC analysis

Equipment: Waters HPLC System, LC 600 pump and UV detector (Model 2487).

Column: Spherisorb S100Ds1, 25 cm×4.6 mm

Column temperature: 25° C.

Flow rate: 1.0 ml/min

Detection wavelength: UV250ₙₘ

Mobile phase: Methanol:Water (13:7)

Preparation of CRS solution: Weight accurately 15 mg of Schizandrol A to a 50 ml volumetric flask and dissolve with methanol to the volume to produce a solution with 0.3 mg Schizandrol A/per ml.

Preparation of test solutions: Place 0.25 g of raw material powder (Trough No. 3 sieve) into a volumetric flask, add 15 ml of methanol and ultrasonicate (power 250 w, frequency 20 kHz) for 20 minutes. Add methanol to the volume, mix well and filter.

Quantity of injection: Inject 10 μl of CRS solution and 10 μl of test solution, respectively.

Result: See chromatograms in FIGS. 8 and 9. FIG. 8 (the BDS) shows at least 6 identifiable peaks including Schizandrol A at a retention time of about 14/15 minutes. The area under the graph indicates a presence of at least 2% by weight of Schizandrol A. The FIG. 9 chromatogram is a control with the marker alone.

| Specifications for Schizandrol A content (% | |
|---|---|
| w/w) | Result (% w/w) |
| >2.0 | 2.4 |

### Example 12

Hepatitis C patients underwent a double blind, placebo controlled trial. Each patient was given either a sachet of the medicament (Example 2) or a placebo twice a day for a period of 24 weeks.

Primary outcome was assessed using quality of life scores (SF36) and (FSS)

Secondary measures of liver inflammation included measuring the following biochemical activities:
GGT (gamma glutamyl amino transferase),
ALT, (alanine amino transferase) and
AST levels (aspartyl amino transferase)
Total bilirubin levels and
Alkaline phosphotase

Additionally safety was assessed with reference to:
Haemoglobin levels
White blood cell activity
Blood platelet activity
Creatinine activity and
Glucose levels.

The trial demographics are illustrated in the table below:

| | N | Age | Sex (M/F) |
|---|---|---|---|
| Placebo | 20 | 46.6 | 11/9 |
| (Total) | | | |
| Placebo | 14 | 45.9 | 7/7 |
| (Completed) | | | |
| Active | 23 | 49.7 | 15/8 |
| (Total) | | | |
| Active | 14 | 50.5 | 9/5 |
| (Completed) | | | |

The results are most clearly seen with reference to FIGS. 14 to 24.

FIGS. 14 and 15 give the primary outcome results:

Referring to FIG. 14 it will be noted that the patients on the “active” had more vitality and better general health. (high-lighted). They also showed (reading from left to right) better physical functioning (PF), had less bodily pain (BP); exhibited improved mental health (MH) and social functioning (SF) although their physical role (RP) and emotional role (RE) were reduced.

Referring to FIG. 15 the patients on active showed improvements in all nine Fatigue Symptom Score measurements.

The secondary measures indicative of reduced liver inflammation are shown in FIGS. 16 to 19.

Referring to FIG. 16 (patients on active) it can be seen that their enzyme activity was reduced (relative to base line) indicating reduced inflammation.

In contrast patients on placebo (FIG. 17) showed no such improvement.

This is most clearly illustrated in FIG. 18 where those on active show a sustained improvement with time whilst those on placebo showed either no, or a worsening, change.

Indeed, as can be seen from FIG. 19—compare FIG. 19a a patient on active with FIG. 19b a patient on placebo, patients on active showed an improvement in all indicators measured, particularly ALT, AST and GGT levels.

The fact that a beneficial effect is seen with a wide range of markers is indicative of the broader potential of this combination in treating liver inflammation associated with a number of conditions including:
I. Liver inflammation associated with hepatitis B virus;
II. Liver inflammation associated with alcohol abuse;
III. Metabolic disorders associated with the liver including for example, diabetes and metabolic syndrome X;
IV. Fatty liver;
V. Treating patients who are non responsive to immuno modulatory/antiviral combination therapies such as, interferon/ribovarin;
VI. As an adjunct therapy to combination therapies such as, interferon/ribovarin
VII. HCV associated liver disease;
VIII. Hepatitis, fibrosis, cirrhosis and hepatocellular carcinoma;
IX. Treatment to reduce raised liver enzyme levels associated with chemotherapy.

That such a drug might be used in combination therapies such as with interferon/ribivarin is supported by the positive safety data obtained. In this regard interferon/ribivarin despite being the gold standard treatment effect does not have a good profile.

Thus the medicament of the invention has:
no noticeable effect on haemoglobin levels (FIG. 20);
no noticeable effect on white blood cell levels (FIG. 21),
no noticeable effect on platelet levels (FIG. 22);
no noticeable effect on creatinine levels (FIG. 23) and
no noticeable effect glucose levels (FIG. 24).

1. The use of a botanical drug or dietary supplement consisting essentially of botanical raw materials, botanical drug substances or botanical ingredients from each of:
(a) The fruit of Silybum marianum;
(b) The root of Astragalus membranaceus var mongholicus or Hedysarum polybotlys;
(c) The root of Salvia miltiorrhiza, Salvia bowleyana or Salvia przewalskii; and
(d) The fruit of Schisandra chinensis or Schisandra sphenanthera in the manufacture of a medicament for use in the treatment or prevention of one or more of the following:
i) Liver inflammation associated with hepatitis B virus;
ii) Liver inflammation associated with alcohol abuse;
iii) Metabolic disorders associated with the liver;
iv) Fatty liver;
v) Treating patients who are non responsive to immuno-modulatory/antiviral combination therapies such as, interferon/ribovarin;
vi) As an adjunct therapy to combination therapies such as, interferon/ribovarin
vii) HCV associated liver disease;
viii) Hepatitis; fibrosis, cirrhosis or hepatocellular carcinoma
ix) Treatment to reduce raised liver enzyme levels associated with chemotherapy.

2. The use of a botanical drug or dietary supplement as claimed in claim 1 wherein each species is present in an amount, relative to the total weight of all of the botanical raw materials, botanical drug substances or botanical ingredients, as follows:
a) Silybum spp. from 22-48%;
b) Astragalus spp. or Hedysarum spp. from 20-63%;
c) Salvia spp. from 13-48%; and
d) Schisandra spp. from 2-19%.

3. The use of a botanical drug or dietary supplement as claimed in claim 2 wherein each species is present in an amount as follows:
(a) Silybum spp. from 30-40%;
(b) Astragalus or Hedysarum spp. from 20-30%;
(c) Salvia spp. from 20-30%; and
(d) Schisandra spp. from 7.5-15%.

4. The use of a botanical drug or dietary supplement as claimed in claim 2 wherein each species is present in an amount as follows:
(a) Silybum spp. 35.3% plus or minus 10%;
(b) Astragalus or Hedysarum spp. 26.5% plus or minus 10%;
(c) Salvia spp. 26.5% plus or minus 10%; and
(d) Schisandra spp. 11.7% plus or minus 10%.

5. The use of a botanical drug as claimed in claim 1 which consists essentially of botanical drug substances.

6. The use of a botanical drug as claimed in claim 5 further comprising excipients.

7. The use of a botanical drug as claimed in claim 5 wherein the botanical drug substances comprise total extracts derived from each of the botanical raw materials.

8. The use of a botanical drug as claimed in claim 5 wherein the botanical drug substances comprise one or more defined extract fractions derived from each of the botanical raw materials.

9. The use of a botanical drug as claimed in claim 5 in which the botanical drug substances are standardised extracts.

10. The use of a botanical drug as claimed in claim 9 wherein the botanical drug substance from the Silybum spp. is standardised against a marker of silybin.

11. The use of a botanical drug as claimed in claim 9 wherein the botanical drug substance from the Silybum spp. comprises at least 30% by weight silybin and isosilybin when calculated by HPLC method.

12. The use of a botanical drug as claimed in claim 9 wherein the standardized extract of the Silybum spp. is a brownish yellow powder which is or has:
(i) no less than 30% silybin by HPLC;
(ii) no more than 0.5% soluble in pentane;
(iii) a sulphated ash content of no more than 1%;
(iv) a heavy metal content of no more than 100 ppm;
(v) a residnal organic solvent content of no more than 1% ethanol, no more than 0.01% ethyl acetate and no more than 0.01% hexane;
(vi) a bacterial content of no more than 1000 cfu/g; and
(vii) a fungal content of no more than 100 cfu/g.

13. The use of a botanical drug as claimed in claim 9 wherein the botanical drug substance from the Astragalus spp. is standardised against a marker of Astragaloside IV.

14. The use of a botanical drug as claimed in claim 13 wherein the botanical drug substance from the Astragalus spp. comprises at least 0.4% by (weight) Astragaloside IV as calculated by HPLC method.

15. The use of a botanical drug as claimed in claim 13 wherein the botanical drug substance from the Astragalus spp. has a TLC chromatographic fingerprint substantially as illustrated in FIG. 1 or a HPLC fingerprint substantially as illustrated in FIG. 4.

16. The use of a botanical drug as claimed in claim 13 wherein the standardized extract of Astragalus spp. is a pale yellow powder which is or has:
(i) no less than 0.4% Astragaloside IV;
(ii) a total ash content of no more than 5%;
(iii) an acid insoluble ash content of no more than 2%; and
(iv) a microbial total viable aerobic count of no more than of 1000 cfulg.

17. The use of a botanical drug as claimed in claim 9 wherein the botanical drug substance from the Salvia spp. is standardised against a marker of Tanshinone II A.

18. The use of a botanical drug as claimed in claim 17 wherein the botanical drug substance from the Salvia spp. comprises at least 1.5% by (weight) of Tanshinone as calculated by HPLC method.

19. The use of a botanical drug as claimed in claim 17 wherein the botanical drug substance from the Salvia spp. has a TLC chromatographic fingerprint substantially as illustrated in FIG. 2 or a HPLC fingerprint substantially as illustrated in FIG. 6.

20. The use of a botanical drug as claimed in claim 17 wherein the standardized extract of the Salvia spp. is a dark red powder which is or has:
(i) no less than 1.5% Tanshinone IIA by HPLC;
(ii) a total ash content of no more than 5%;
(iii) an acid insoluble ash content of no more than 2%; and
(iv) a microbial total viable aerobic count of no more than of 1000 cfu/g.

21. The use of a botanical drug as claimed in claim 9 wherein botanical drug substance from the Schisandra spp. is standardised against a marker of Schizandrol A.

22. The use of a botanical drug as claimed in claim 21 wherein the botanical drug substance from the Schisandra spp. comprises at least 2.0% by weight Schizandrol A by HPLC method.

23. The use of a botanical drug substance, as claimed in claim 21 wherein the botanical drug substance from the Schisandra spp. has a TLC chromatographic fingerprint substantially as illustrated in FIG. 3 or a HPLC fingerprint substantially as illustrated in FIG. 8.

24. The use of a botanical drug substance as claimed in claim 22 wherein the standardised extract of Schisandra spp. is a brownish red powder which is or has:
(i) no less than 20% Schisandrol A;
(ii) a total ash content of no more than 5%;
(iii) an acid insoluble ash content of no more than 2%; and
(iv) a microbial total viable aerobic count of no more than of 1000 cfu/g.

25. The use of a botanical drug as claimed in claim 9 wherein each standardised extract is a dried ethanolic extract.

26. The use of a botanical drug as claimed in claim 9 wherein the Silybum spp. is extracted according to a process substantially as illustrated in FIG. 10.

27. The use of a botanical drug as claimed in claim 9 wherein the Astragalus 10 spp. is extracted according to a process substantially as illustrated in FIG. 11.

28. The use of a botanical drug as claimed in claim 9 wherein the: Salvia spp. is extracted according to a process substantially as illustrated in FIG. 12.

29. The use of a botanical drug as claimed in claim 9 wherein the Schisandra spp. is extracted according to the process substantially as illustrated in FIG. 13.

30. The use of a botanical drug as claimed in claim 9 which is provided in a unit dosage form.

31. The use of a botanical drug as claimed in claim 30 wherein said unit dosage form is a suspension powder mixture.

32. The use of a botanical drug as claimed in claim 31 further comprising as excipients:
a) one or more gellants or thickeners comprising at least one xanthum gum having a particle size distribution such that 100% by weight of the particles pass a 60 mesh sieve, 95% by weight of the particles pass a 80 mesh sieve and 70% by weight of the particles pass a 200 mesh sieve,
b) one or more fillers; and
c) one or more wetting agents and or surfactants.

33. The use of a botanical drug as claimed in claim 32 wherein the xanthan gum has a molecular weight of from 3.5 to 4.0×10⁶.

34. The use of a botanical drug as claimed in claim 32 wherein the wetting agent is a polyethylene glycol or macrogol.

35. The use of a botanical drug as claimed in claim 30 further comprising one or more of a disintegrating agent, a lubricant, a sweetening agent, a flavouring agent and a viscosifying agent.

36. The use of a botanical drug as claimed in claim 30 which is packaged in a sachet.

37. The use of a botanical drug as claimed in claim 30 which is packaged with a dispensing container.

38. The use of a botanical drug, as claimed in claim 37 wherein the dispensing container has a sealable lid.

39. The use of a botanical drug as claimed in claim 9 comprising in a unit dose:
i) 0.200 g to 0.250 g of a botanical drug substance from a Silybum spp. (equivalent to 12 g to 15 g of botanical raw material);
ii) 0.585 g to 1.95 g of a botanical drug substance from a Astragalus spp. (equivalent to 9 g to 30 g of botanical raw material);
iii) 0.225 g to 0.375 g of a botanical drug substance from a Salvia spp. (equivalent to 9 g to 15 g f botanical raw material) and
iv) 0.150 g to 0.600 g of a botanical drug substance from a Schisandra spp. (equivalent to 1.5 g to 6 g of botanical raw material).

40. A method of treating a patient to alleviate or prevent one or more of the following:
i) Liver inflammation associated with hepatitis B virus;
ii) Liver inflammation associated with alcohol abuse;
iii) Metabolic disorders associated with the liver;
iv) Fatty liver;
v) Treating patients who are non responsive to immuno-modulatory/antiviral combination therapies such as, interferon/ribovarin;
vi) As an adjunct therapy to combination therapies including, interferon/ribovarin;
vii) HCV associated liver disease;
viii) Hepatitis; fibrosis, cirrhosis or hepatocellular carcinoma;
ix) Treatment to reduce raised liver enzyme levels associated with chemotherapy comprising administering to the patient a composition consisting essentially of botanical raw materials, botanical drug substances or botanical ingredients from each of:
(a) The fruit of Silybum marianum;
(b) The root of Astragalus membranaceus var mongholicus or Hedysarum polybotrys;
(c) The root of Salvia miltiorrhiza, Salvia bowleyana or Salvia przewalskii; and
(d) The fruit of Schisandra chinensis or Schisandra sphenanthera.
